(19) United States
(12) Patent Application Publication
(10) Pub. No.: US 2026/0185157 A1
(43) Pub. Date: Jul. 2, 2026

(54) SEQUENCING WITH CONCATEMERIZATION

(71) Applicant: ULTIMA GENOMICS, INC., Fremont, CA (US)

(72) Inventors: Ron Saar DOVER, Fremont, CA (US); Xi LONG, Fremont, CA (US); Florian OBERSTRASS, Redwood City, CA (US); Robert ONO, Fremont, CA (US); William SCHULKINS, Fremont, CA (US); Seth STERN, Menlo Park, CA (US)

(73) Assignee: ULTIMA GENOMICS, INC., Fremont, CA (US)

(21) Appl. No.: 19/179,090

(22) Filed: Apr. 15, 2025

Related U.S. Application Data
(63) Continuation of application No. PCT/US2023/035508, filed on Oct. 19, 2023.
(60) Provisional application No. 63/417,946, filed on Oct. 20, 2022.
(60) Provisional application No. 63/581,542, filed on Sep. 8, 2023.

(51) Int. Cl. C12Q 1/6874

(52) U.S. Cl. CPC C12Q 1/6874

(57) ABSTRACT

Provided herein are systems and methods for amplifying nucleic acid samples, immobilizing such amplified products onto substrates, and sequencing the amplified products on the substrates. The amplified products may comprise concatemers.

### CROSS-REFERENCE

This application is a continuation of International Patent Application No. PCT/US2023/035508 filed Oct. 19, 2023, which claims the benefit of U.S. Provisional Patent Application Nos. 63/417,946, filed Oct. 20, 2022, and 63/581,542, filed Sep. 8, 2023. Each of the foregoing applications is incorporated by reference herein in their entirety for all purposes.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious effort.

### SUMMARY

Provided herein are systems and methods for amplifying nucleic acid samples, immobilizing such amplified products onto substrates, and sequencing the amplified products on the substrates. The amplified products may comprise concatemers.

In some embodiments, the method for sequencing concatemers comprises providing a substrate having a plurality of concatemers immobilized thereto; extending sequencing primers hybridized to the plurality of concatemers in a plurality of extension steps; and detecting signals, or lack thereof, from the substrate during or subsequent to at least a subset of the plurality of extension steps to determine sequencing reads corresponding to the plurality of concatemers. The substrate is rotated about a rotational axis (i) during or subsequent to one or more extension step(s) of the plurality of extension steps, or (ii) during the detecting, or during both (i) and (ii).

In some embodiments, extending of the sequencing primers comprises providing, in the extension step, a plurality of nucleotides under conditions sufficient to incorporate the plurality of nucleotides, where at least a subset of the plurality of nucleotides is labeled, and where the signals, or lack thereof, to determine sequencing reads are indicative of incorporation of labeled nucleotides of the plurality of nucleotides.

In some embodiments, all of the nucleotides provided in the extension step are labeled.

In some embodiments, at most 50% of the nucleotides provided in the extension step are labeled. In some embodiments, at most 10% of the nucleotides provided in the extension step are labeled.

In some embodiments, some nucleotides provided in the extension step comprise a single canonical base type selected from A, T, U, G, or C. In some embodiments, some nucleotides provided in the extension step comprises two or three canonical base types. In some embodiments, some nucleotides provided in the extension step comprises four canonical base types.

In some embodiments, some labeled nucleotides provided in the extension step are detectable at a same wavelength or wavelength range. In some embodiments, some labeled nucleotides provided in the extension step are detectable at different wavelengths or wavelength ranges.

In some embodiments, some nucleotides provided in the extension step are reversibly terminated nucleotides. In some embodiments, some nucleotides provided in the extension step are non-terminated nucleotides.

In some embodiments, the substrate used for sequencing is patterned. In some embodiments, the substrate used for sequencing is unpatterned. In some embodiments, the substrate used for sequencing is aminated. In some embodiments, the substrate used for sequencing comprises azido groups.

In some embodiments, the substrate used for sequencing comprises a polyethylene glycol (PEG) spacer. In some embodiments, the substrate used for sequencing comprises a layer of a PEG spacer. In some embodiments, the PEG spacer comprises a multi-arm PEG molecule. In some embodiments, the PEG spacer comprises a methyl terminated PEG molecule.

In some embodiments, the substrate used for sequencing comprises a plurality of surface primers that are covalently bound to the substrate. In some embodiments, a plurality of concatemers is hybridized to the surface primers, and the surface primers comprise sequencing primers.

In some embodiments, the substrate used for sequencing is rotated about a rotational axis during or subsequent to one or more extension step(s) of the plurality of extension steps. In some embodiments, the substrate used for sequencing is rotated about a rotational axis during the detecting. In some embodiments, the substrate used for sequencing is rotated about a rotational axis (i) during or subsequent to one or more extension step(s) of the plurality of extension steps, and (ii) during the detecting step.

In some embodiments, the method for sequencing comprises, prior to providing the substrate used for sequence, loading a plurality of concatemers on the substrate to immobilize the plurality of concatemers on the substrate. In some embodiments, the substrate used for sequencing comprises a plurality of surface primers that are hybridized to the plurality of concatemers, where the plurality of surface primers comprises sequencing primers.

In some embodiments, the substrate used for sequencing does not have surface primers.

In some embodiments, the method for sequencing comprises contacting sequencing primers to a plurality of concatemers subsequent to immobilizing the plurality of concatemers on the substrate used for sequencing.

In some embodiments, the method for sequencing comprises contacting sequencing primers to a plurality of concatemers prior to loading the plurality of concatemers on the substrate used for sequencing.

In some embodiments, the method for sequencing comprises prior to loading the plurality of concatemers on the substrate, amplifying a plurality of circular templates in solution to generate the plurality of concatemers, where the plurality of circular templates comprises different nucleic acid template inserts. In some embodiments, the method of sequencing comprises prior to amplifying the plurality of circular templates, circularizing a plurality of linear templates comprising the different nucleic acid template inserts using splint molecules and ligating the respective two ends of the plurality of linear templates to generate the circular templates. In some embodiments, the splint molecules are used to amplify the plurality of circular templates.

In some embodiments, amplifying comprises rolling circle amplification (RCA).

In some embodiments, the method for sequencing comprises, prior to providing the substrate used for sequencing, loading a plurality of circular templates on the substrate, where the substrate comprises a plurality of surface primers; hybridizing the plurality of circular templates to the plurality of surface primers; and using the plurality of surface primers to amplify the plurality of circular templates on the substrate to generate the plurality of concatemers immobilized to the substrate, where the plurality of circular templates comprises different nucleic acid template inserts.

In some embodiments, the method for sequencing comprises, prior to providing the substrate used for sequencing, loading a plurality of circular templates on the substrate, where the plurality of circular templates are hybridized to a plurality of primers that are conjugated to reactive moieties, and where the substrate comprises additional reactive moieties configured to couple to the reactive moieties, configured to bind the plurality of circular templates to the substrate, and using the plurality of primers to amplify the plurality of circular templates on the substrate, configured to generate the plurality of concatemers immobilized to the substrate, where the plurality of circular templates comprises different nucleic acid template inserts.

In some embodiments, the method for sequencing comprises circularizing a plurality of linear templates comprising different nucleic acid template inserts using splint molecules and ligating the respective two ends of the plurality of linear templates to generate circular templates.

In some embodiments, the method for sequencing comprises, prior to providing a substrate for sequencing, loading a plurality of linear templates on the substrate, where the substrate comprises a plurality of surface primers; hybridizing the plurality of linear templates to the plurality of surface primers; ligating the respective two ends of the plurality of linear templates to generate a plurality of circular templates; and using the plurality of surface primers to amplify the plurality of circular templates to generate the plurality of concatemers immobilized to the substrate, where the plurality of linear templates comprises different nucleic acid template inserts.

In some embodiments, the method for sequencing comprises extending the sequencing primers hybridized to the plurality of concatemers to generate a plurality of second concatemers, hybridizing second sequencing primers to the plurality of second concatemers, extending the second sequencing primers in a second plurality of extension steps, and detecting second signals, or lack thereof, from the substrate during or subsequent to at least a subset of the second plurality of extension steps to determine second sequencing reads corresponding to the plurality of concatemers.

In some embodiments, the method for sequencing comprises processing the sequencing reads and second sequencing reads as paired end reads.

In some embodiments, the method for sequencing comprises amplifying the plurality of concatemers to generate a plurality of second concatemers, hybridizing second sequencing primers to the plurality of second concatemers, extending the second sequencing primers in a second plurality of extension steps, and detecting second signals, or lack thereof, from the substrate during or subsequent to at least a subset of the second plurality of extension steps to determine second sequencing reads corresponding to the plurality of concatemers.

In some embodiments, the plurality of concatemers is amplified via amplification primers covalently bound to the substrate. In some embodiments, the plurality of concatemers is amplified via amplification primers that are not covalently bound to the substrate. In some embodiments, the plurality of concatemers is covalently bound to the substrate. In some embodiments, the plurality of concatemers is not covalently bound to the substrate. In some embodiments, the plurality of concatemers is bound to the substrate via electrostatic attraction.

In some embodiments, a method for paired end sequencing comprises hybridizing a first primer to a first primer binding site on a template molecule; extending the first primer through a first region of the template molecule, where the extending comprises alternatively adding nucleotides and detecting incorporation of nucleotides; extending the first primer through a second region of the template molecule, thereby producing a copied template molecule, where the extending comprises adding nucleotides of at least one base type and, at one or more time points, not detecting incorporation of nucleotides; denaturing the copied template molecule from the template molecule; hybridizing a second primer to a second primer binding site on the copied template molecule; and extending the second primer through a first region of the copied template molecule, wherein the extending comprises alternatively adding nucleotides and detecting incorporation of nucleotides.

In some embodiments, the method for paired end sequencing comprises extending the second primer through a second region of the copied template molecule, where the extending comprises adding nucleotides of at least one base type and, at one or more time points, not detecting incorporation of nucleotides.

In some embodiments of the method for paired end sequencing, the step of extending the first primer through a second region of the template molecule comprises, in one or more extension steps, adding nucleotides of two base types. In some embodiments of the method for paired end sequencing, the step of extending the first primer through a second region of the template molecule comprises, in one or more extension steps, adding nucleotides of three base types. In some embodiments of the method for paired end sequencing, the step of extending the first primer through a second region of the template molecule comprises, in one or more extension steps, adding nucleotides of four base types.

In some embodiments of the method for paired end sequencing, a sequence of the first region of the template molecule is determined from detection of nucleotide incorporation in the extending of the first primer through the first region of the template molecule and by at least one detection of nucleotide incorporation in the extending of the second primer through the first region of the copied template molecule.

In some embodiments of the method for paired end sequencing, a sequence of the second region of the template molecule is determined from detection of nucleotide incorporation in the extending of the second primer through the first region of the copied template molecule and by at least one detection of nucleotide incorporation in the extending of the first primer through the first region of the template molecule.

In some embodiments of the method for paired end sequencing, each detection determines a base type of the respective incorporated nucleotide. In some embodiments of the method for paired end sequencing, each detection comprises a confidence value of a respective nucleotide incorporation.

In some embodiments of the method for paired end sequencing, the first primer binding site is at the 3′ end of the template molecule. In some embodiments of the method for paired end sequencing, the second primer binding site is at the 3′ end of the copied template molecule.

In some embodiments of the method for paired end sequencing, the template molecule and the copied template molecule are each single-stranded.

In some embodiments of the method for paired end sequencing, the nucleotides added during the extending the first primer through the first region of the template molecule and extending the second primer through a first region of the copied template molecule comprises reversibly terminated, labeled nucleotides.

In some embodiments of the method for paired end sequencing, the nucleotides added during the extension of the first primer through the second region of the template molecule and during the extension of the second primer through the second region of the copied template molecule comprise a first subset of unlabeled nucleotides and a second subset of labeled nucleotides.

In some embodiments of the method for paired end sequencing, the nucleotides added during the extension of the first primer through the second region of the template molecule and during the extension of the second primer through the second region of the copied template molecule comprise labeled nucleotides.

In some embodiments of the method for paired end sequencing, the nucleotides added during the extension of the first primer through the second region of the template molecule and during the extension of the second primer through the second region of the copied template molecule comprise unterminated nucleotides.

In some embodiments of the method for paired end sequencing, at least a subset of the nucleotides added during the extension of the first primer through the first region of the template molecule and during the extension of the second primer through the first region of the copied template molecule comprise unlabeled and/or unterminated nucleotides.

In some embodiments of the method for paired end sequencing, wherein the extension of the first primer through the first region of the template molecule and the extension of the second primer through the first region of the copied template molecule comprise, after detecting incorporation of nucleotides, cleaving reversible terminators from incorporated nucleotides.

In some embodiments, a method for loading concatemers on a substrate for sequencing comprises depositing a plurality of bead assemblies onto the substrate. The substrate comprising a plurality of individually addressable locations and a bead assembly of the plurality of bead assemblies comprises a circular template and a bead having surface primers. The circular template bound to the bead via one of the surface primers. The plurality of bead assemblies immobilized on the plurality of individually addressable locations on the substrate. The method further comprises using the surface primers to amplify the circular template to generate a plurality of first stage concatemers and second stage concatemers immobilized to the substrate via the bead. The method further comprises sequencing the first stage concatemers or the second stage concatemers immobilized to the substrate.

In an aspect, provided is a method for loading concatemers on a substrate for sequencing, comprising: (a) depositing a plurality of bead assemblies to a substrate comprising a plurality of individually addressable locations, wherein a bead assembly of the plurality of bead assemblies comprises (i) a bead comprising surface primers and (ii) a circular template, wherein the circular template is bound to the bead via one of the surface primers, wherein the plurality of bead assemblies are immobilized on the plurality of individually addressable locations on the substrate; (b) using the surface primers, amplifying the circular template to generate a plurality of first stage concatemers and second stage concatemers immobilized to the substrate via the bead; and (c) sequencing the first stage concatemers or the second stage concatemers immobilized to the substrate.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIGS. 3A-3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule.

FIGS. 7A-7C illustrate multiplexed stations in a sequencing system.

FIG. 8 illustrates a computer system that is programmed or otherwise configured to implement methods provided herein.

FIGS. 9A-9C illustrate example splint-assisted circularization workflows.

FIGS. 10A-10N illustrate different workflows for amplifying circular template molecules and sequencing concatemers. FIG. 10A illustrates a workflow in which concatemers are deposited on a patterned substrate; FIG. 10B illustrates a workflow in which bead-bound concatemers are deposited on a patterned substrate; FIG. 10C illustrates a workflow in which concatemers are deposited on an un-patterned substrate; FIG. 10D illustrates a workflow in which bead-bound concatemers are deposited an un-patterned substrate; FIG. 10E illustrates a workflow in which circularized templates are deposited on a patterned substrate; FIG. 10F illustrates a workflow in which circularized templates are deposited on an un-patterned substrate; FIG. 10G illustrates a workflow in which bead-bound circularized templates are deposited on a substrate via click chemistry; FIG. 10H illustrates a workflow in which dendrimer-bound concatemers are deposited on a substrate; FIG. 10I illustrates a workflow in which click chemistry bead-bound concatemers are deposited on a substrate; FIG. 10J illustrates a workflow in which bead-bound circularized templates are deposited on a patterned substrate; FIG. 10K illustrates a workflow in which concatemers are deposited on a substrate with pads; FIG. 10L illustrates a workflow in which concatemers are deposited on an unpatterned substrate; FIG. 10M illustrates a workflow in which linear templates or circular templates are deposited on a substrate and rolling circle amplification is performed on surface; and FIG. 10N illustrates a workflow in which circular templates are deposited on a substrate and attached to the surface via click chemistry and rolling circle amplification is performed on surface.

FIGS. 11A-11C illustrate nitride-functionalization of a surface.

FIGS. 11D-11K illustrate surface preparation and loading schemes.

FIGS. 11L and 11M illustrate template loading and on-surface amplification on surfaces with either surface oligos or surface oligos and PEG molecules.

FIG. 12 illustrates a schematic for paired end sequencing.

FIG. 13A illustrates probe assay results for adding ethylene carbonate during rolling circle amplification; FIG. 13B illustrates probe assay results for adding ethylene carbonate during sequencing primer hybridization.

FIG. 14A illustrates sequencing results from concatemers generated from performing on surface rolling circle amplification; FIGS. 14B-14C illustrate sequencing results from concatemers generated from performing rolling circle amplification in solution and subsequently immobilized to the substrate.

FIG. 15 illustrates in panel (A) an example 3 Arm PEG-Azide molecule, in panel (B) an example 4 Arm PEG-Azide molecule, and in panel (C) an example DBCO-PEG-DBCO molecule.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. In some cases, a blocking group may be an azidomethyl or disulfide blocking group. Examples of 3′-O-blocked reversible terminators include, for example, 3′-O-(2-nitrobenzyl) reversible terminators, 3′-ONH₂ reversible terminators, 3′-O-(2-cyanoethyl) reversible terminators, 3′-O-allyl reversible terminators, and 3′-O-aziomethyl reversible terminators. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp, and the “lightning terminator” developed by Michael L. Metzker et al. A reversible terminator may comprise a blocking group in a linker (e.g., a cleavable linker) and/or dye moiety of a nucleotide analog. The blocking groups may be attached to the nucleotide via a cleavable linker. In some instances, the blocking groups may comprise a reporter moiety (e.g., dye moiety). Alternatively, the reporter moiety may be attached to the nucleotide at a different location (e.g., at a nucleobase) via an independent linker. In some instances, the linker for the blocking group and the linker for the dye may be the same type of linker and/or otherwise be cleavable via the same stimulus (e.g., cleaving agent). Cleavable linkers can include, for example, disulfide linkers and fluoride-cleavable linkers. The reversibly terminated nucleotide may be unblocked, such as by cleaving the blocking group (e.g., using a cleaving reagent or irradiation), to reverse the termination. Unblocking may be facilitated by introducing one or more cleaving agents. The cleaving agent may be dependent on the unblocking group present. For example, reducing agents may be used to cleave disulfide bonds or other reductive cleavage groups. Reducing agents include, but are not limited to, phosphine compounds, water soluble phosphines, nitrogen containing phosphines and salts and derivatives thereof, dithioerythritol (DTE), dithiothreitol (DTT) (cis and trans isomers, respectively, of 2,3-dihydroxy-1,4-dithiolbutane), 2-mercaptoethanol or β-mercaptoethanol (BME), 2-mercaptoethanol or amino ethanethiol, glutathione, thioglycolate or thioglycolic acid, 2,3-dimercaptopropanol and tris(2-carboxyethyl)phosphine (TCEP), tris(hydroxymethyl)phosphine (THP) and p-[tris(hydroxymethyl)phosphine] propionic acid (THPP). A phosphine reagent may include triaryl phosphines, trialkyl phosphines, sulfonate containing and carboxylate containing phosphines and derivatized water soluble phosphines. In another example, such as for 2-cyanoethyl blocking groups and/or cyanoethyl ester linkers, fluoride ions (e.g., solution comprising tetrabutylammonium fluoride (TBAF), etc.) can be used as cleaving agents. See, e.g., Diana C. Knapp et al., Fluoride-Cleavable, Fluorescently Labelled Reversible Terminators: Synthesis and Use in Primer Extension, 17 CHEM. EUR. J. 2903-15 (2011), and Diana C. Knapp et al., Fluorescent Labeling of (Oligo) Nucleotides by a New Fluoride Cleavable Linker Capable of Versatile Attachment Modes, 21 BIOCONJUGATE CHEM. 1043-55 (2010), which are entirely incorporated herein by reference.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:
(e.g., [A T G C A T G C A T G A T G A T G A T G C A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order and can be expressed in units of flow cycles. For example, where [A TG C] is identified as a 1^{st} flow cycle, and [A T G] is identified as a 2^{nd} flow cycle, the flow order of [A T G C A T G C A T G A T G A T G A T G C A T G C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle, 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11 (2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65 (2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3): 443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3): 403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity, and as such sequences would generally be considered “identical.”

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD), Hybrid TDI, complementary metal oxide semiconductor (CMOS) pseudo TDI device, or TDI line-scan camera.

### Sample Processing Methods

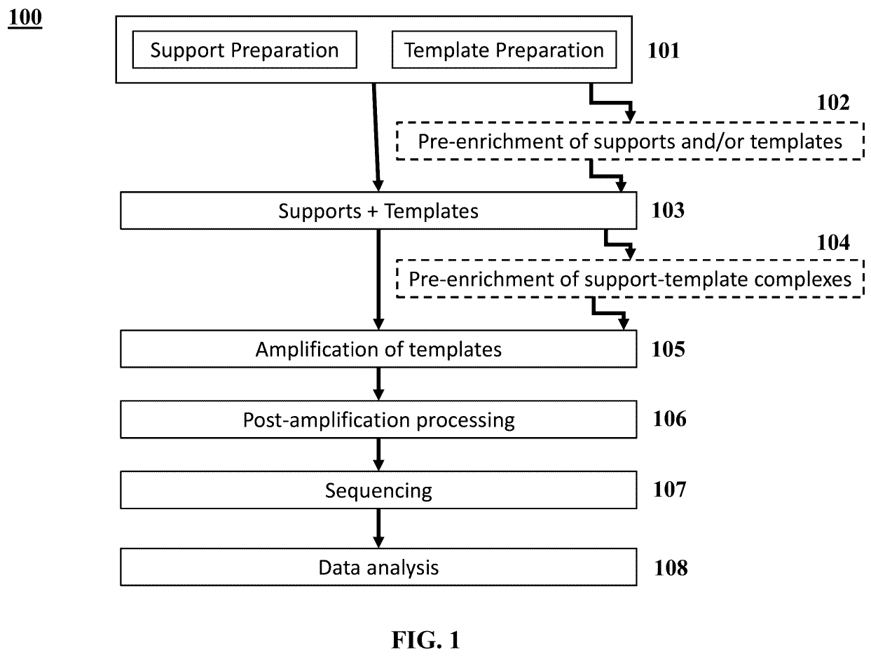

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture or be coupled to a template nucleic acid (or processed template nucleic acid). For example, the support may comprise the capture sequence, a primer sequence, a barcode sequence, a sample index sequence, a unique molecular identifier (UMI), a flow cell adapter sequence, an adapter sequence, a binding sequence for any molecule (e.g., splint, primer, template nucleic acid, capture sequence, etc.), or any other functional sequence useful for a downstream operation, or any combination thereof. The oligonucleotide may be single-stranded, double-stranded, or partially double-stranded.

A support may comprise one or more capture entities, where a capture entity is configured for capture by a capturing entity. A capture entity may be coupled to an oligonucleotide coupled to the support. A capture entity may be coupled to the support. For example, the capturing entity may comprise streptavidin (SA) when the capture moiety comprises biotin. In another example, the capturing entity may comprise a complementary capture sequence when the capture entity comprises a capture sequence (e.g., a capture oligonucleotide that is complementary to the complementary capture sequence). In another example, the capturing entity may comprise an apparatus, system, or device configured to apply a magnetic field when the capture entity comprises a magnetic particle. In another example, the capturing entity may comprise an apparatus, system, or device configured to apply an electrical field when the capture entity comprises a charged particle. In some instances, the capturing entity may comprise one or more other mechanisms configured to capture the capture entity. A capture entity and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively or in addition, a single capturing entity may be capable of associating with multiple capture entities. The capture entity may be capable of linking to a nucleotide. Chemically modified bases comprising biotin, an azide, cyclooctyne, tetrazole, and a thiol, and many others are suitable as capture entities. The capture entity/capturing entity pair may be any combination. The pair may include, but is not limited to, biotin/streptavidin, azide/cyclooctyne, and thiol/maleimide. It will be appreciated that either of the pair may be used as either the capture entity or the capturing entity. In some instances, the capturing entity may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., UDG, RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethyleneglycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adapter sequence of a template nucleic acid and a second subset of oligonucleotides configured to capture a second adapter sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to said support species. When attaching template nucleic acids to supports, only a template nucleic acid comprising a given adapter sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to said support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adapter region of a template nucleic acid or an intermediary primer sequence which can subsequently bind to a complementary nucleic acid sequence within an adapter region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in U.S. Patent Pub. No. 20220042072A and International Patent Pub. No. WO2022040557A2, each of which is entirely incorporated herein by reference for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adapter oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adapter sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adapter sequence, the adapter sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprises capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprises cleavable moieties. A cleavable moiety may be strategically placed based on a desired downstream amplification workflow, for example.

In some examples, a library of insert sequences are processed to provide a population of template sequences with identical configurations, such as with identical sequences and/or locations of one or more functional sequences. For example, a population of template sequences may comprise a plurality of nucleic acid molecules each comprising an identical first adapter sequence ligated to a same end. In some examples, a library of insert sequences are processed to provide a population of template sequences with varying configurations, such as with varying sequences and/or locations of one or more functional sequences. For example, a population of template sequences may comprise a first subset of nucleic acid molecules each comprising an identical first adapter sequence at a first end, and a second subset of nucleic acid molecules each comprising an identical second adapter sequence at the second end, where the second adapter sequence is different form the first adapter sequence. In some instances, a population of template sequences with varying configurations (e.g., varying adapter sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adapter sequence) is isolated from a mixture comprising template nucleic acids that do not have the distinct configuration. Alternatively, a template nucleic acid population may be provided to comprise substantially uniform configurations. In some cases, the capture entit(ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit(ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets in an emulsion during emulsion PCR (ePCR or emPCR), or in wells. Emulsion PCR methods are described in further detail in U.S. Patent Pub. No. 20220042072A1 and International Patent Pub. No. WO2022040557A2, each of which is entirely incorporated by reference herein.

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. No. 10,900,078, U.S. Patent Pub. No. 20210079464A1, and International Patent Pub. No. WO2022040557A2, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Any sequencing method described elsewhere herein may be used. In some cases, sequencing by synthesis (SBS) is performed.

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. Alternatively, only three of the four bases may be labeled with a different dye (yielding different optical signals). The different dyes may be interrogated in 3 channels, and incorporation of the fourth base may be determined from a lack of or background signal detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g., [A T G C]), where each nucleotide is not terminated, and where each base is labeled with a same dye (yielding same frequency optical signals). With each flow, other sequencing reagents, e.g., sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in U.S. Patent Pub. Nos. 20200326327A1, 20210354126A1, 20210079464A1, and 2023/0279487A1 and International Patent Pub. No. WO2023/192403A2, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

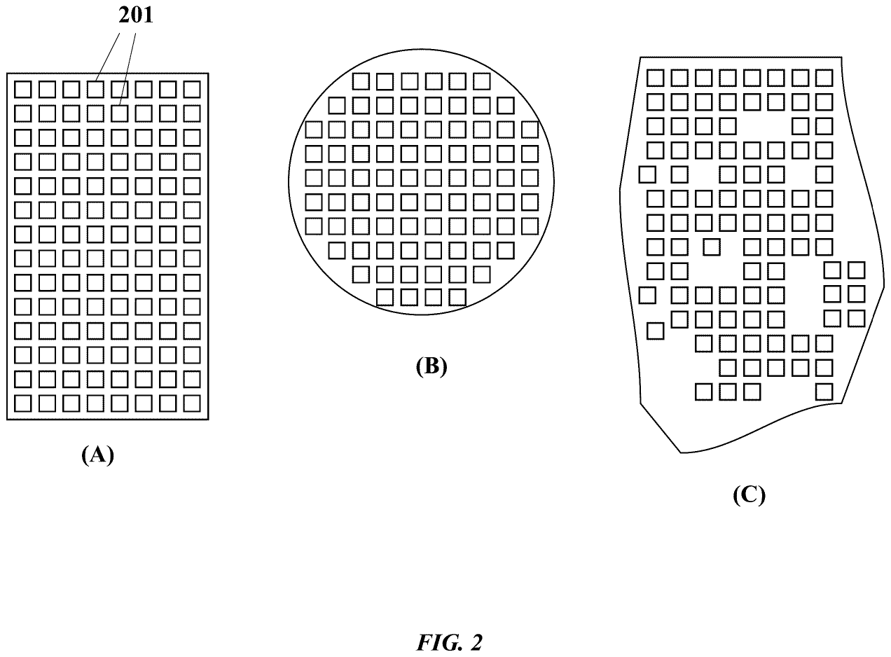

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

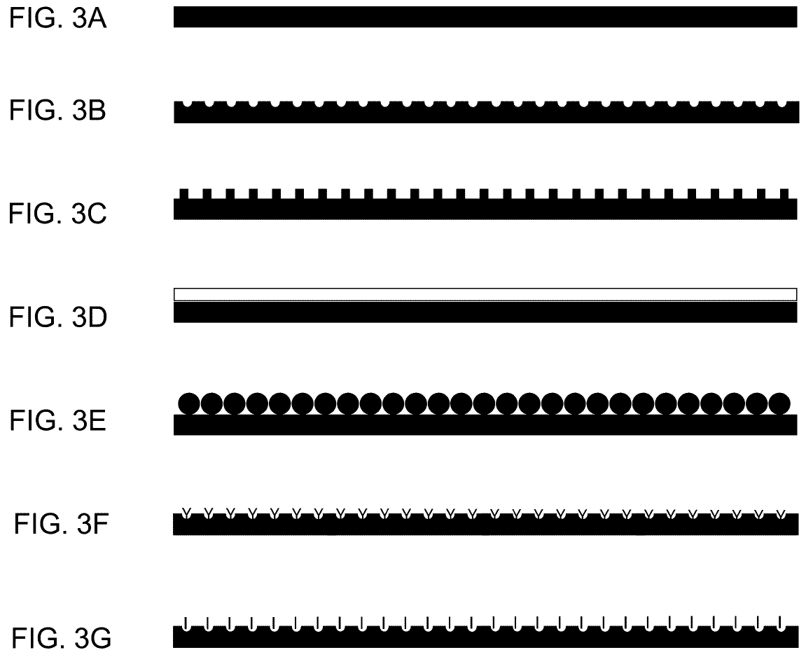

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

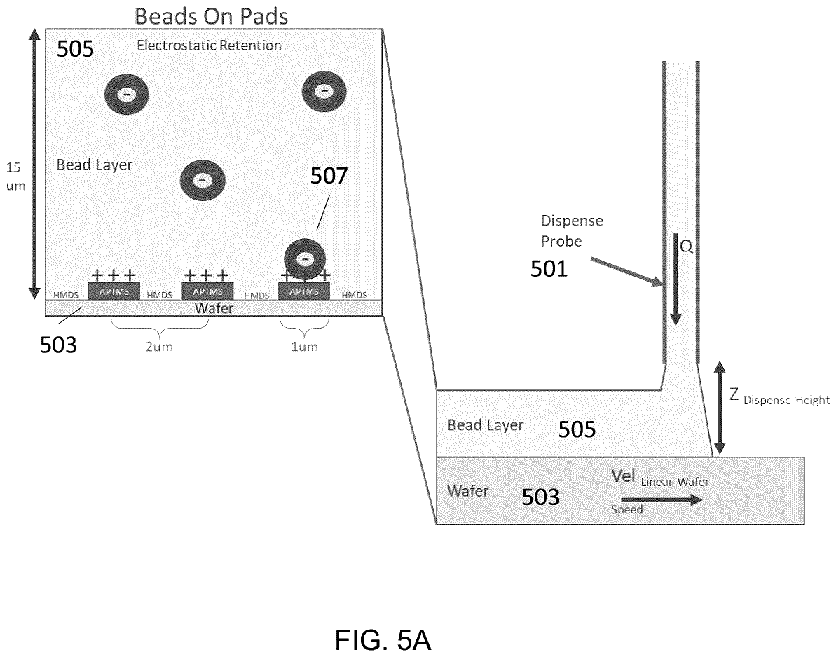

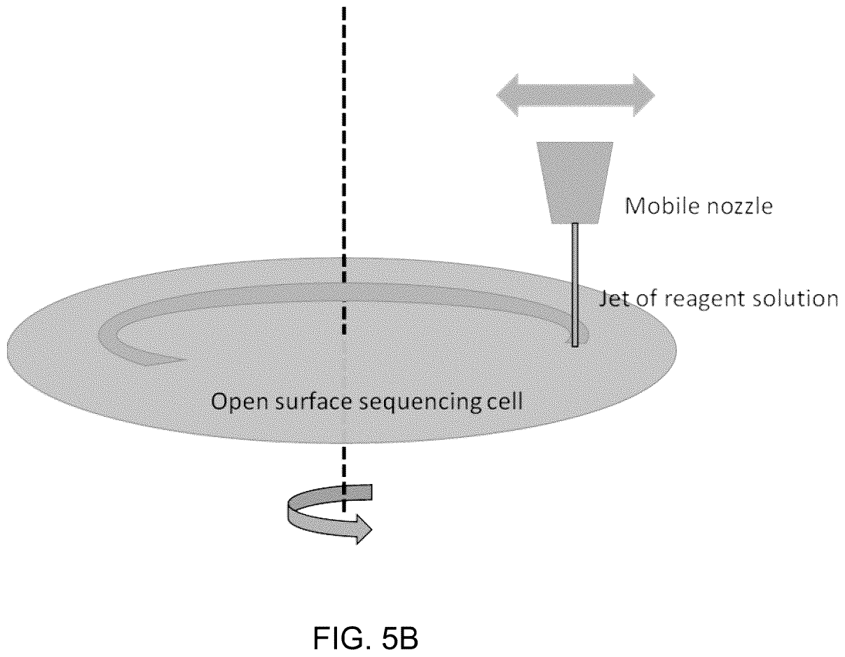

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

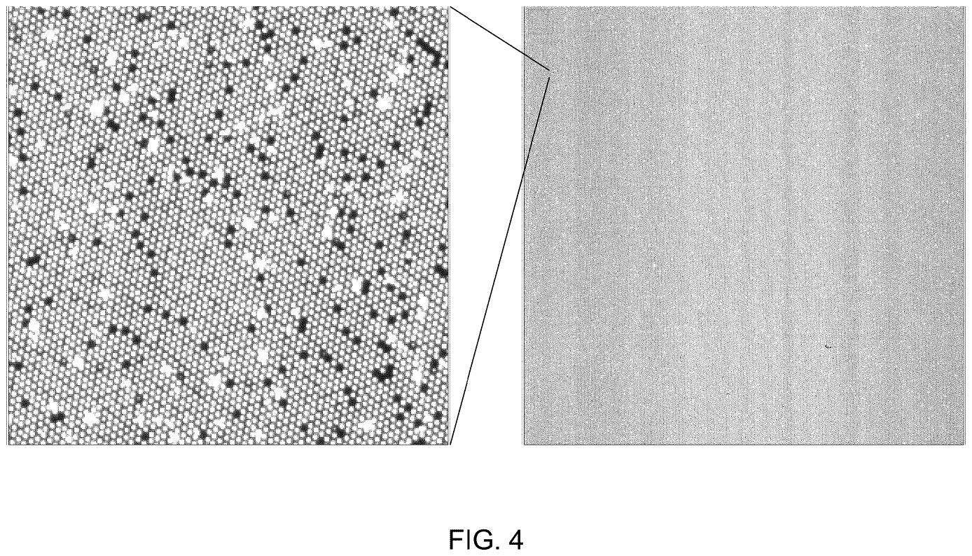

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar functions by high-speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate

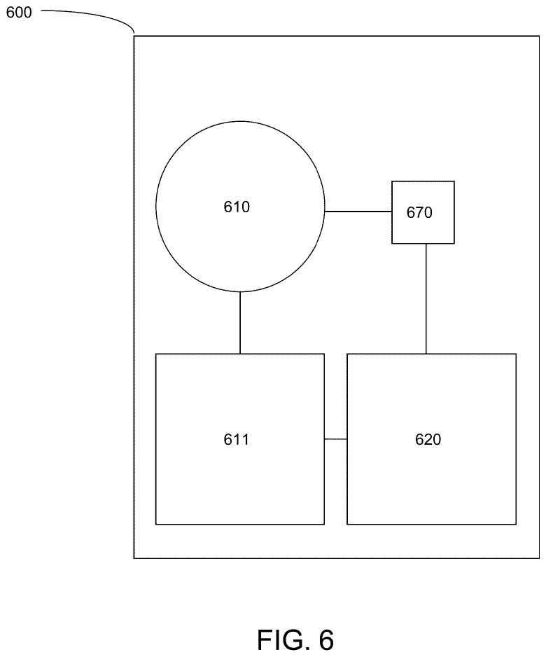

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in U.S. Patent Pub. No. 20210354126A1, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion. Alternatively, a sample environment system may comprise a sample processing environment defined by a chamber and a lid plate, where the lid plate is in contact with the chamber and completely covers and closes the chamber opening.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (C), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

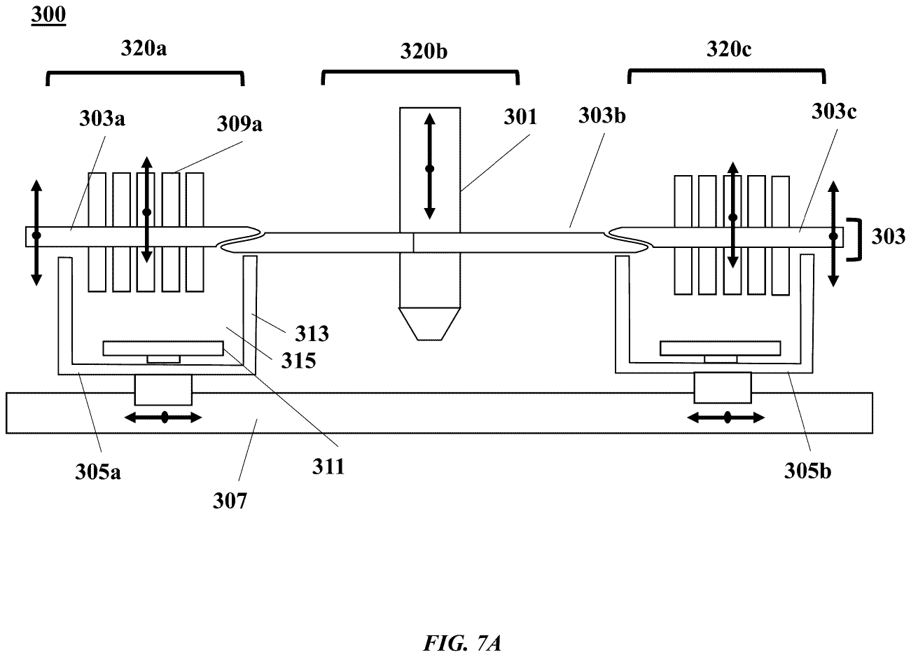

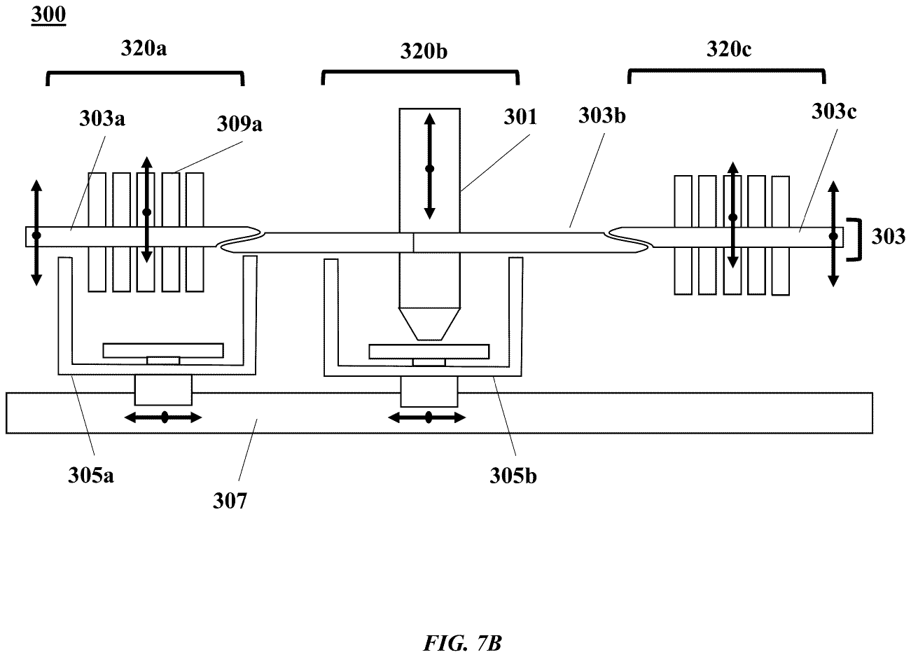

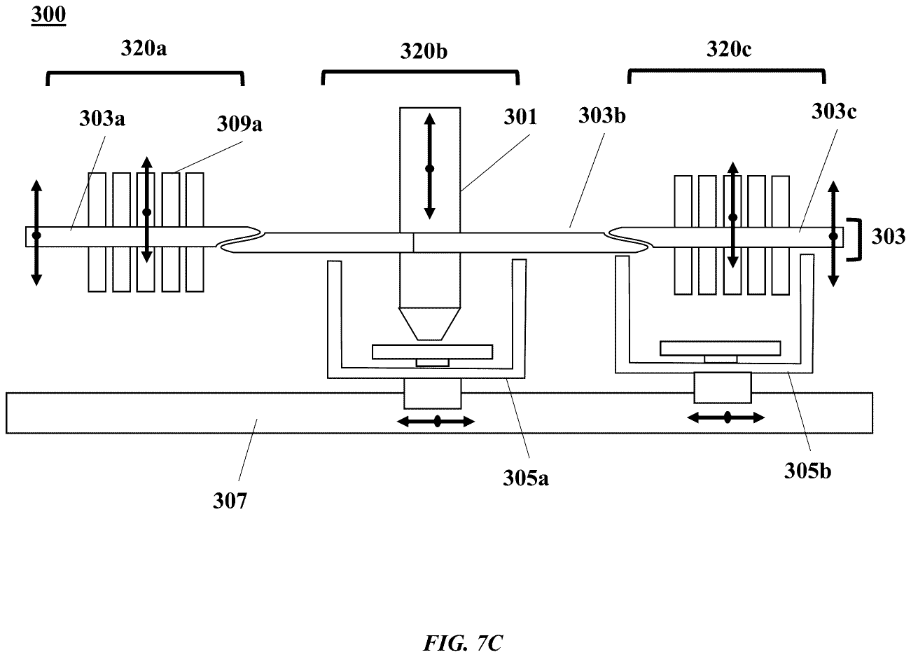

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

Provided are high throughput sequencing systems that comprise multiple stations. In some instances, a station of the sequencing system, and/or operation performed therein, may be controlled independent of other operations and/or independent of other stations in the sequencing system. In some instances, two or more stations of the sequencing system may be controlled together and/or substantially simultaneously, such as with a single set of instructions.

For example, the sequencing system may comprise one or more of a sample station, a substrate station, a sample loading station, a reagent station, a processing station, a detection station, a diluent station, a controlling station, a power station, and an instructions station. In some cases, the system may comprise fewer stations. For example, one or more stations described above may not be included. In some cases, the system may comprise one or more additional stations. In some cases, one or more stations described above may be merged into a single station or split into more stations. A sample station may be configured to receive one or more nucleic acid samples into the system and configured to supply a nucleic acid sample of the nucleic acid sample(s) to a substrate in the sample loading station. A substrate station may be configured to receive one or more substrates (e.g., into a rack) into the system and configured to supply a substrate to the sample loading station. A sample loading station may be configured to dispense a nucleic acid sample of the nucleic acid sample(s) onto a surface of a substrate. A reagent station may be configured to receive reagents in a first reservoir or a second reservoir and configured to supply the reagents to the processing station, where the reagent may be supplied from a first reservoir or a second reservoir or both. A processing station may be configured to bring a nucleic acid molecule of the nucleic acid sample immobilized on the surface of the substrate into contact with a reagent to sequence the nucleic acid molecule. A detection station may be configured to collect one or more signals from the nucleic acid molecule, or derivatives thereof (e.g., extending primer) to sequence the nucleic acid molecule. As described elsewhere herein, a substrate may transfer between the processing station and detection station without leaving its sample processing environment (e.g., enclosed by lid and chamber) by moving a modular sample processing environment between the two stations. In some cases, the substrate may also transfer from the sample loading station to the processing and/or detection station without leaving its sample processing environment by moving a modular sample processing environment between the two stations. In some cases, only the substrate (e.g., loaded with the sample) may be moved from the sample loading station to the processing and/or detection station by using a sample handler to transfer the substrate from a chuck on the sample loading station to a chuck within a sample processing environment in the processing and/or detection station. A diluent station may be configured to dilute the reagents that are dispensed to the substrate by providing a diluent (e.g., deionized or distilled water) from a diluent reservoir or diluent supply prior to or during dispensing of the reagents in the processing station. A controlling station may comprise one or more processors, individually or collectively, programmed to execute any user instructions or operations of the sequencing system. The one or more processors may be operably coupled to various components of the different stations to execute the operations. A power station may be configured to supply power to one or more stations. An instructions station may be configured to receive and communicate user instructions or system instructions to the controlling station. The instructions station may comprise a user interface.

The nucleic acid sequencing systems and optical systems described herein (or any elements thereof) may be combined in a variety of architectures.

### Sequencing Concatemers

Provided herein are devices, systems, methods, compositions, and kits for amplification and sequencing of nucleic acid samples with circularization and/or concatemerization. Amplification methods provided herein may generate concatemers. In some cases, amplification may be performed in a single stage. In some cases, amplification may be performed in multiples stages, such as two stages or more. Amplification may comprise rolling circle amplification (RCA) and/or multiple displacement amplification (MDA). Such devices, systems, methods, compositions, and kits can be applied alternatively or in addition to the various operations described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

### Circularization

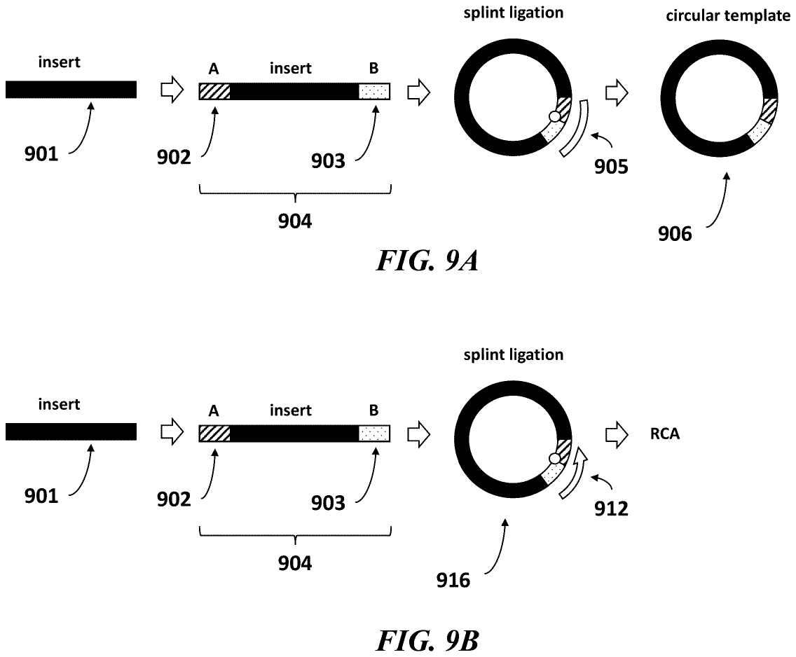

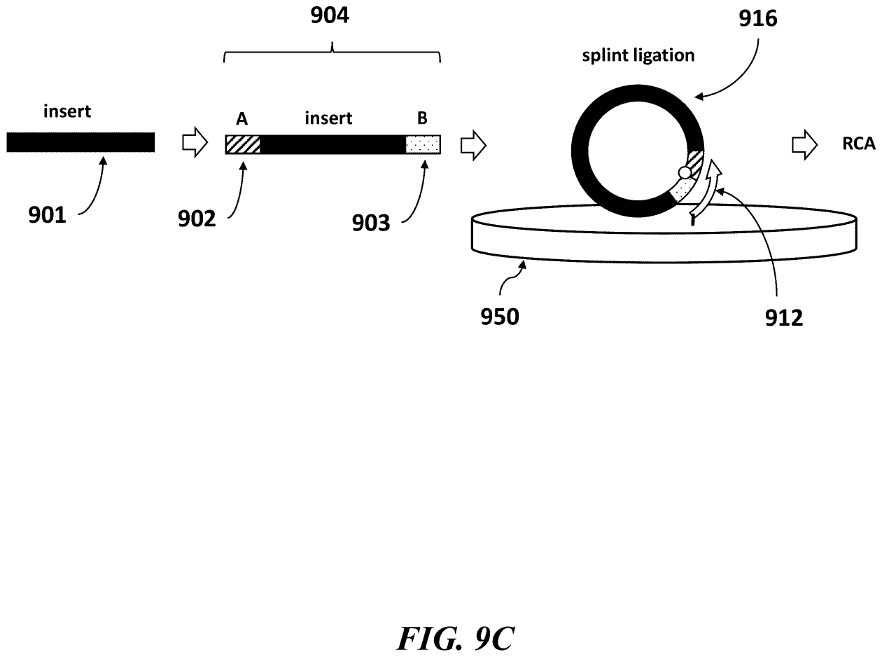

A template nucleic acid may be circularized prior to amplification. FIGS. 9A-9C illustrate an example splint-assisted circularization workflow. A template insert molecule 901 may be ligated to a first adapter 902 (“A”) at a first end and to a second adapter 903 (“B”) at a second end to generate an adapter-ligated template molecule 904. In some cases, the adapter-ligated template may be amplified. Alternatively, or in addition, the adapter-ligated template may be 5′ phosphorylated (or adapters may be phosphorylated in amplification-free protocols) to enable future ligation. As shown in FIG. 9A, a splint molecule 905 may bind to the adapter-ligated template molecule 904, attaching to at least a portion of the first adapter and to at least a portion of the second adapter, to circularize the adapter-ligated template molecule. The two ends of the adapter-ligated template molecule 904 may be ligated and the splint molecule 905 may be removed to generate a circularized template molecule 906. Then, the circularized template molecule 906 may be contacted with a primer to proceed into amplification. As shown in FIG. 9B, in some cases, the splint molecule may comprise a primer 912, and after circularization and ligation the circularized template molecule 916 may proceed directly into amplification, for example by extending the primer 912 using the circularized template molecule 916 as a template. In some cases, the splint molecule may be further attached to a support (e.g., a substrate surface, a bead, etc.) via covalent or non-covalent binding. As shown in FIG. 9C, the splint molecule comprising the primer 912 may be coupled, via covalent or non-covalent binding, to a substrate 950 surface, and the adapter-ligated template molecule 904 may be contacted to the primer 912 on the surface. After circularization and ligation, the circularized template molecule 916 may proceed directly into amplification on the surface, for example by extending the primer 912 using the circularized template molecule 916 as a template.

The splint molecule 905 may comprise a first splint sequence and a second splint sequence adjacent to each other, the first splint sequence complementary to a first sequence of the first adapter 902 and the second splint sequence complementary to a second sequence of the second adapter 903. In cases where a splint molecule comprising the primer 912 is further attached to a support (e.g., a substrate surface, a bead, etc.), the splint molecule may further comprise a third splint sequence enabling such attachment (e.g., where the third splint sequence is complementary to a support-bound oligonucleotide molecule, or where the third splint sequence is bound to another molecule that may bind to a support or to another molecule on the support (e.g., covalently bound to biotin)). In some cases, the splint molecule may comprise a moiety (e.g., molecule), such as a reactive moiety, that can bind to another moiety. An adapter molecule (e.g., first adapter 902, second adapter 903) may comprise an adapter sequence and/or one or more functional sequences. In some cases, the adapter sequence or functional sequence, or its complement, may be configured to attach to an oligonucleotide molecule (e.g., surface primer) coupled to the surface of a support and/or a substrate for downstream applications. The first and second adapters may comprise different sequences. The first and second adapters may comprise the same sequence.

In some cases, after binding the splint molecule to the adapter-ligated template molecule, there may be a gap between the two ends of the template which is filled (e.g., with aid of a polymerase) before ligation. The circularization may be performed with a splint (splint-assisted) or without a splint. Ligases that can be used include Taq DNA ligase, T3 DNA ligase, T4 DNA ligase, T7 DNA ligase, E. coli DNA ligase, TS2126 RNA ligase, Circligase™ ssDNA ligase, Thermophage™ ssDNA ligase, SplintR® ligase, etc.

### Concatemers on Open Substrates

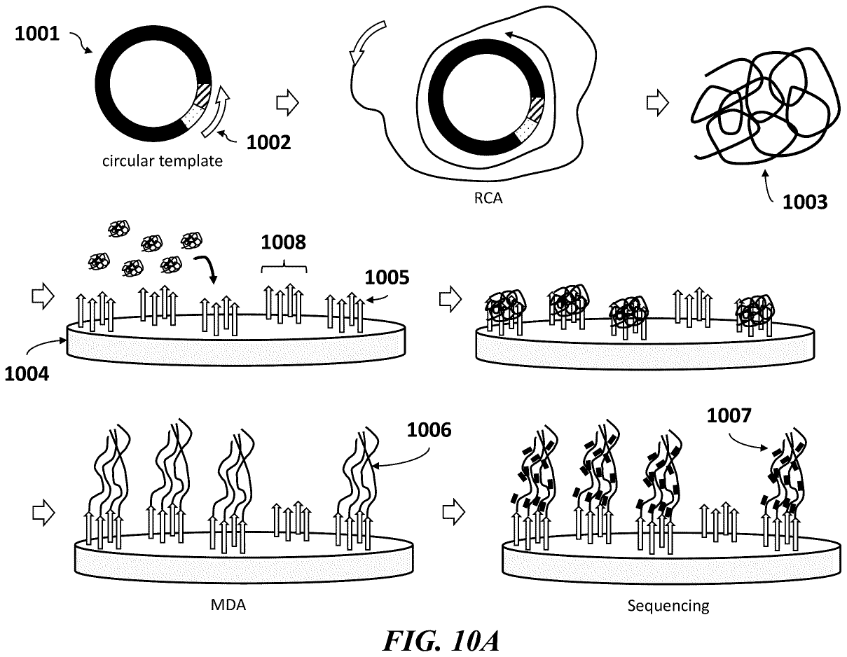

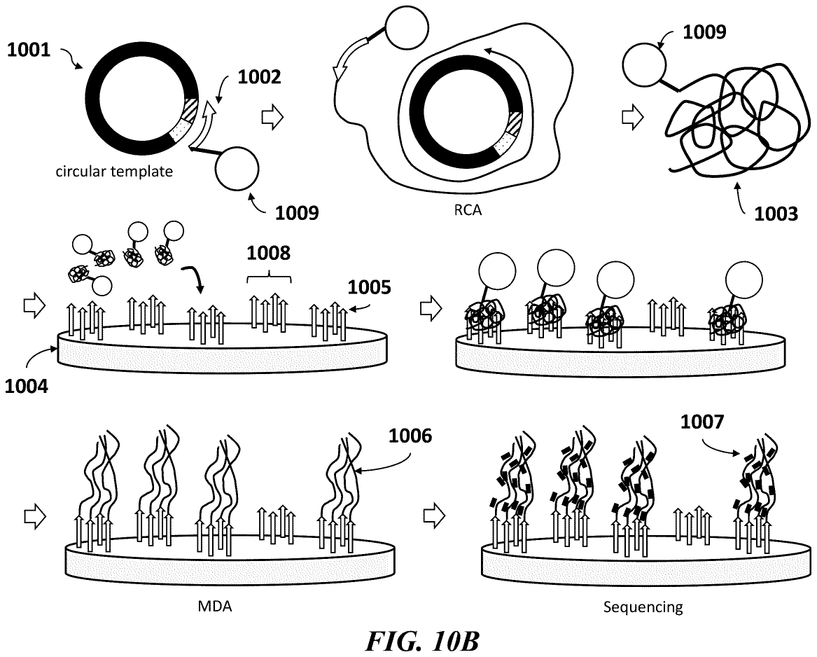

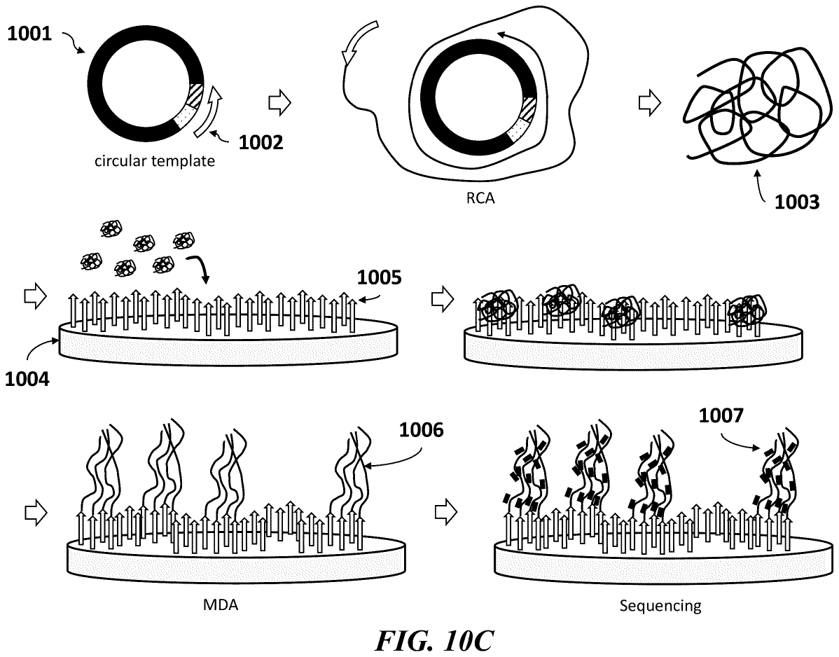

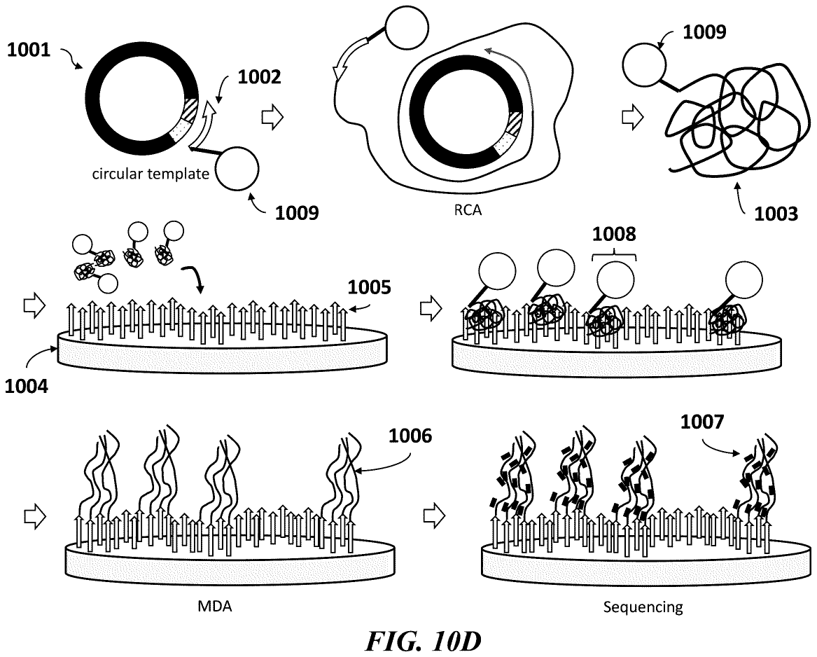

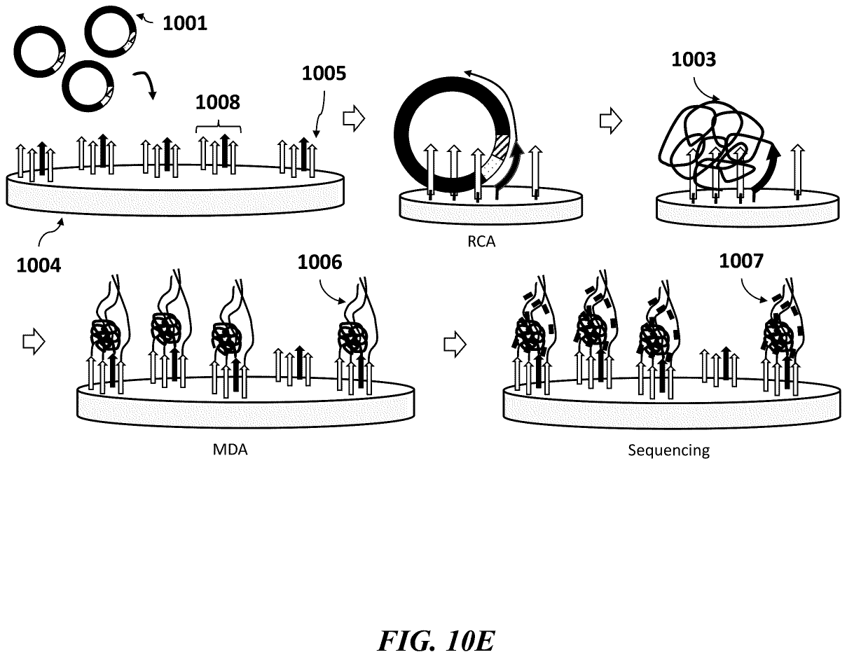

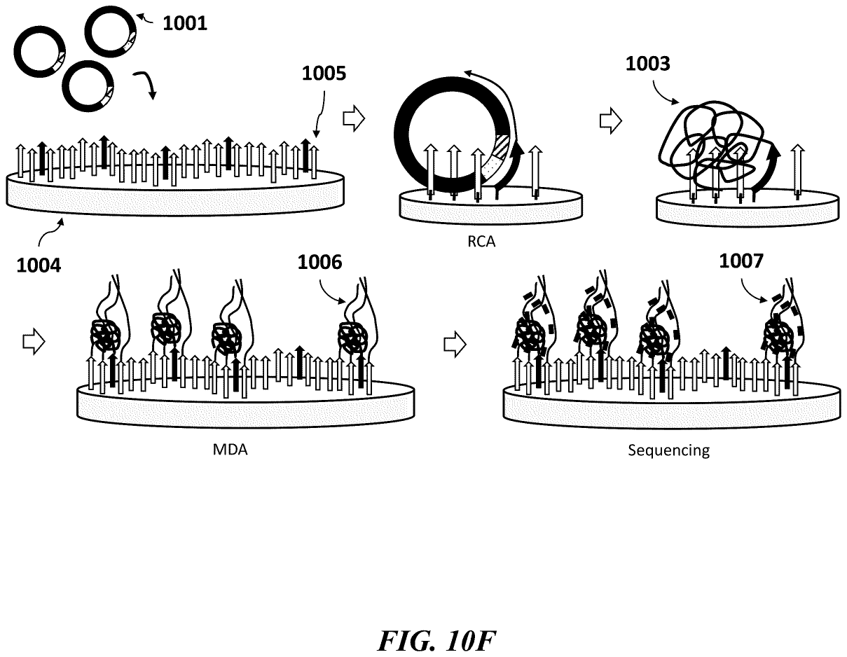

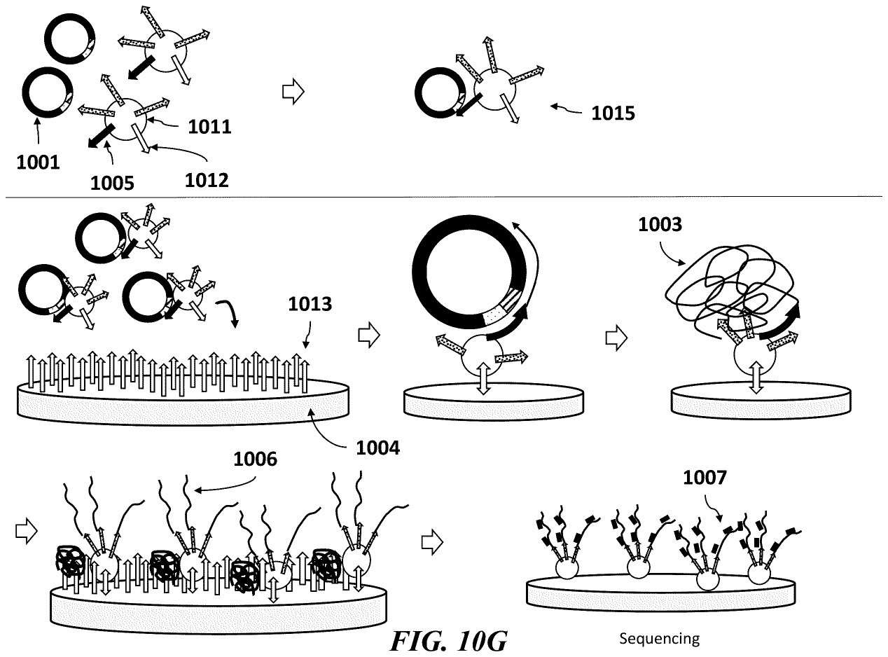

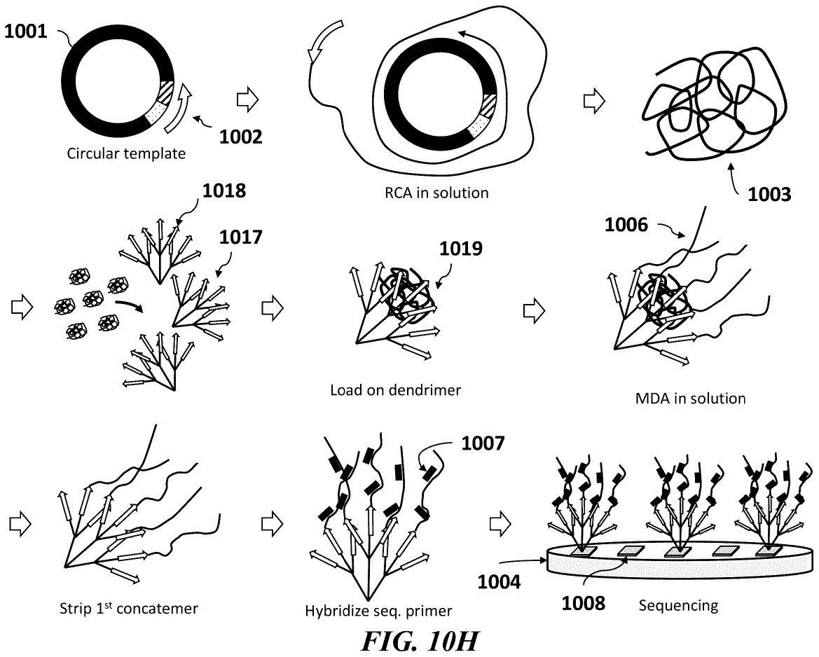

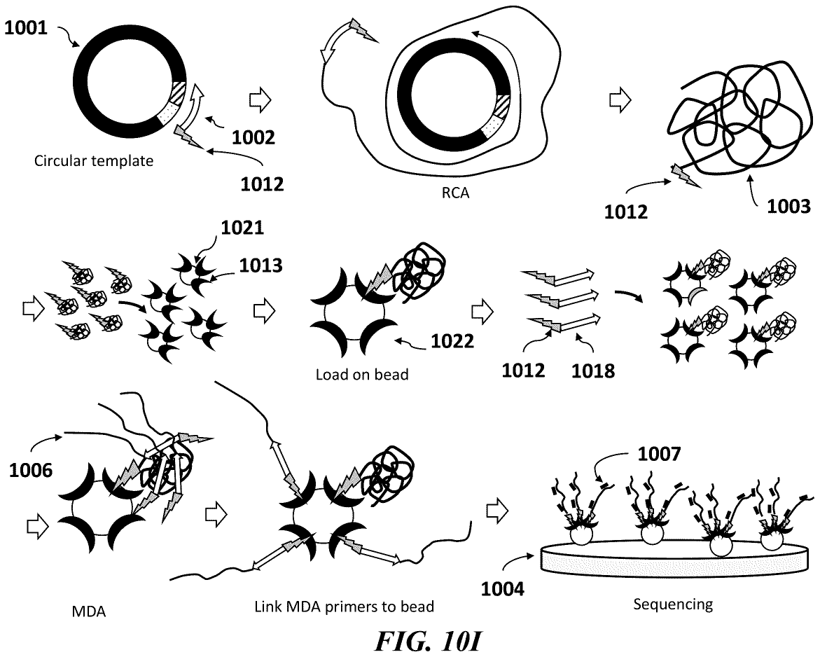

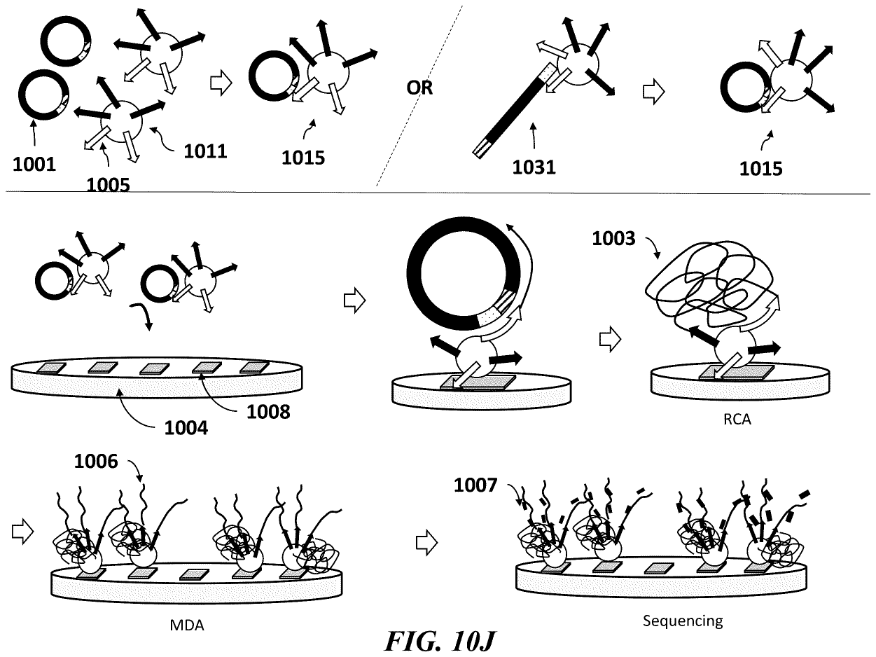

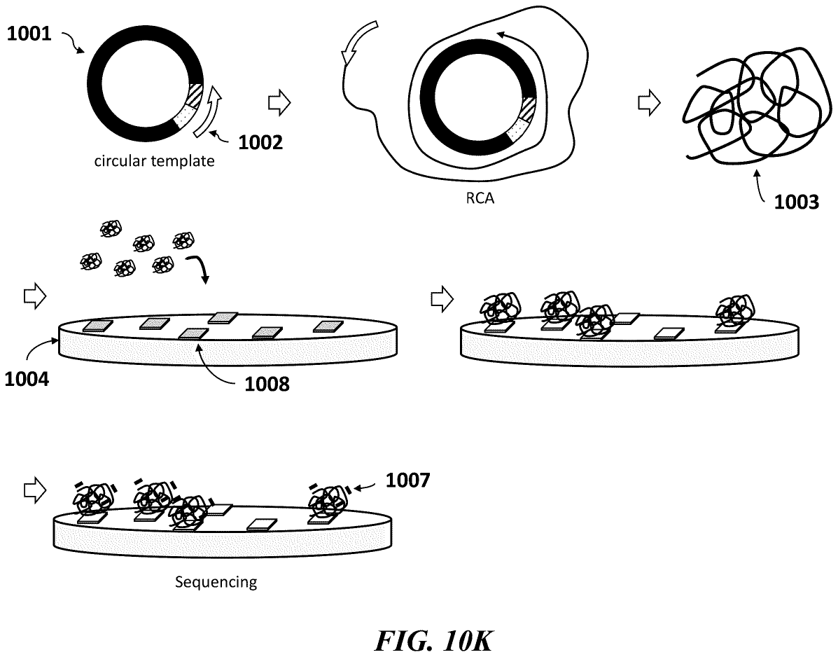

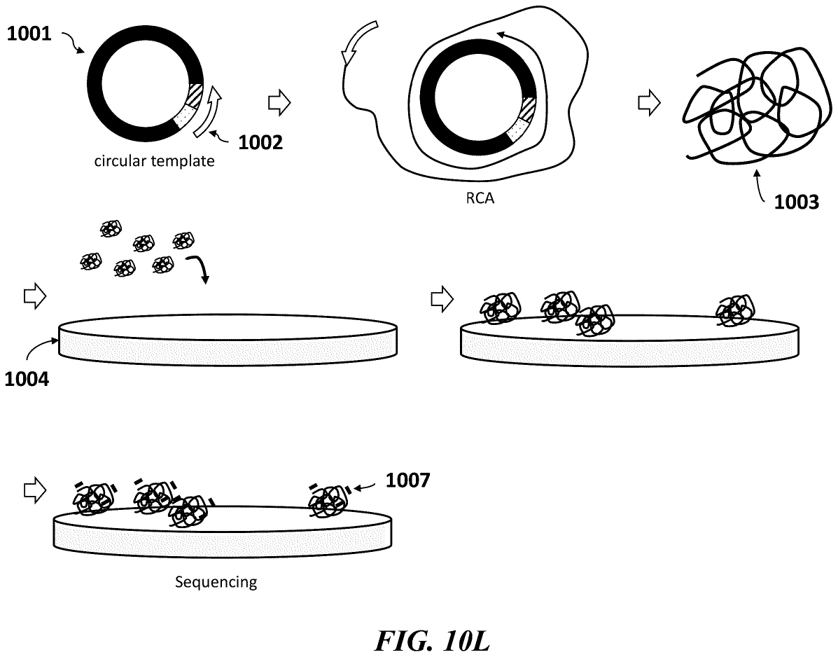

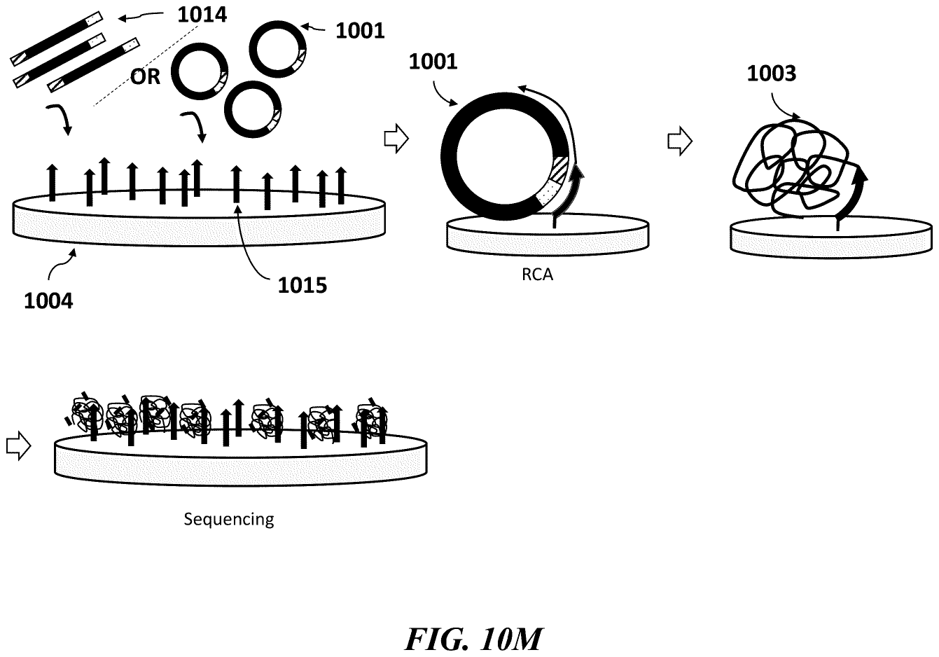

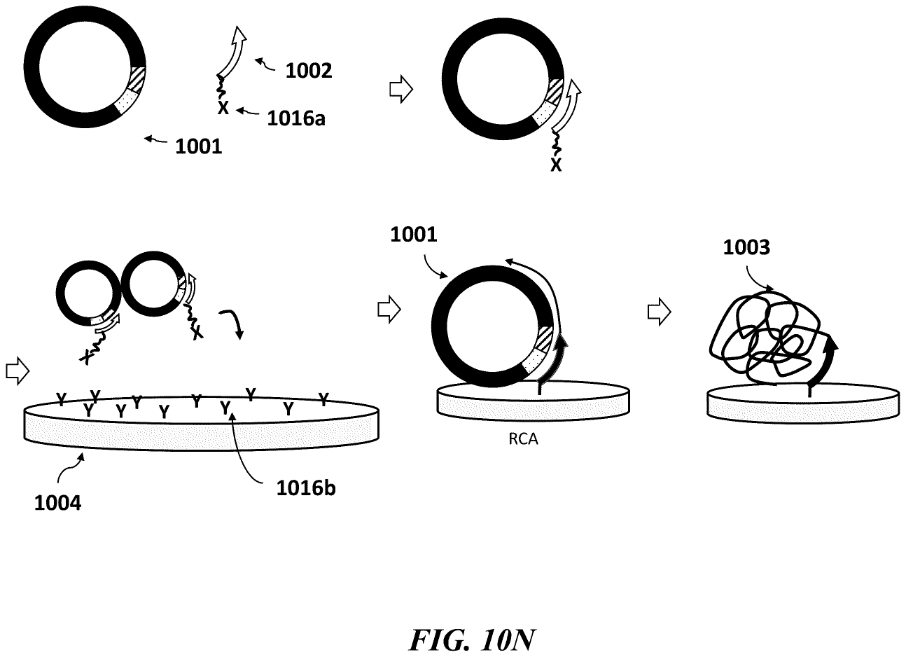

FIGS. 10A-10N illustrate different workflows for amplifying circular template molecules and sequencing concatemers. In these workflows, circular template molecules may be prepared according to any of the circularization workflows described herein (e.g., FIGS. 9A-9C). Accordingly, a circular template 1001 may comprise a first adapter, a template insert, and a second adapter. A concatemer may comprise at least two repeating oligonucleotide units. In an example, an oligonucleotide unit comprises a first adapter, a template insert, and a second adapter, in the listed order or other orders. A concatemer may be a nanoball.

In FIG. 10A, in a first stage amplification, a circular template 1001 may be amplified in solution using a solution primer 1002, such as via RCA to generate a first stage concatemer 1003. The solution primer 1002 may bind to the circular template 1001 at the first adapter and/or second adapter. The first stage concatemer 1003 and other first stage concatemers generated from the template library may be deposited onto a substrate 1004 patterned with individually addressable locations 1008. Each individually addressable location of a plurality of individually addressable locations on the substrate 1004 may comprise a cluster of surface primers 1005, the individually addressable locations discretely spaced apart from each other. The surface primers 1005 may bind to the sequence(s) of the first stage concatemers corresponding to the first adapter and/or second adapter. After deposition, at most one first stage concatemer (e.g., 1003) may be bound to each individually addressable location. A single first stage concatemer may be bound to multiple surface primers at the individually addressable location. In a second stage amplification, the first stage concatemers (e.g., 1003) may be amplified on the substrate using the surface primers 1005, such as via MDA to generate second stage concatemers 1006 immobilized to the individually addressable locations 1008 of the substrate 1004. It will be appreciated that the surface primers 1005 can include forward and reverse primers, and during second stage amplification, concatemers in the forward and reverse directions can be generated, such as via RCA and/or MDA. The generation of one type of concatemer can propagate the generation of the other type of concatemer and accelerate the amplification process. After the second stage amplification, an individually addressable location may have bound thereto a plurality of second stage concatemers. The second stage concatemers 1006 may then be sequenced on the substrate according to systems and methods described herein, such as by providing a plurality of sequencing primers 1007. The sequencing signals collected from each individually addressable location producing a signal may be attributed to a single distinct template insert. Where one type of concatemer has been sequenced first, the other type of concatemer may additionally be sequenced.

In FIG. 10B, in a first stage amplification, a circular template 1001 may be amplified in solution using a solution primer 1002 coupled to a bead 1009, such as via RCA to generate a first stage concatemer 1003 coupled to a bead 1009. The solution primer 1002 may bind to the circular template 1001 at the first adapter and/or second adapter. The bead-bound first stage concatemer 1003 and other bead-bound first stage concatemers generated from the template library may be deposited onto a substrate 1004 patterned with individually addressable locations 1008. Each individually addressable location of a plurality of individually addressable locations on the substrate 1004 may comprise a cluster of surface primers 1005, the individually addressable locations discretely spaced apart from each other. The surface primers 1005 may bind to the sequence(s) of the first stage concatemers corresponding to the first adapter and/or second adapter. After deposition, at most one bead-bound first stage concatemer (e.g., 1003) may be bound to each individually addressable location. A single bead-bound first stage concatemer may be bound to multiple surface primers at the individually addressable location. In a second stage amplification, the bead-bound first stage concatemers (e.g., 1003) may be amplified on the substrate using the surface primers 1005, such as via MDA to generate second stage concatemers 1006 immobilized to the individually addressable locations 1008 of the substrate 1004. The beads may be washed away. It will be appreciated that the surface primers 1005 can include forward and reverse primers, and during second stage amplification, concatemers in the forward and reverse directions can be generated, such as via RCA and/or MDA. After the second stage amplification, an individually addressable location may have bound thereto a plurality of second stage concatemers. The second stage concatemers 1006 may then be sequenced on the substrate according to systems and methods described herein, such as by providing a plurality of sequencing primers 1007. The sequencing signals collected from each individually addressable location producing a signal may be attributed to a single distinct template insert. Where one type of concatemer has been sequenced first, the other type of concatemer may additionally be sequenced.

In FIG. 10C, in a first stage amplification, a circular template 1001 may be amplified in solution using a solution primer 1002, such as via RCA to generate a first stage concatemer 1003. The solution primer 1002 may bind to the circular template 1001 at the first adapter and/or second adapter. The first stage concatemer 1003 and other first stage concatemers generated from the template library may be deposited onto a substrate 1004 coated with a plurality of surface primers 1005. The surface primers may not be spaced apart in distinct clusters. The surface primers 1005 may bind to the sequence(s) of the first stage concatemers corresponding to the first adapter and/or second adapter. A single first stage concatemer may be bound to multiple surface primers at the individually addressable location. In a second stage amplification, the first stage concatemers (e.g., 1003) may be amplified on the substrate using the surface primers 1005, such as via MDA to generate second stage concatemers 1006 immobilized to the substrate 1004. It will be appreciated that the surface primers 1005 can include forward and reverse primers, and during second stage amplification, concatemers in the forward and reverse directions can be generated, such as via RCA and/or MDA. The second stage concatemers 1006 may then be sequenced on the substrate according to systems and methods described herein, such as by providing a plurality of sequencing primers 1007. The sequencing signals collected from a distinct individually addressable location producing a signal may be attributed to a single distinct template insert. Where one type of concatemer has been sequenced first, the other type of concatemer may additionally be sequenced.

In FIG. 10D, in a first stage amplification, a circular template 1001 may be amplified in solution using a solution primer 1002 coupled to a bead 1009, such as via RCA to generate a first stage concatemer 1003 coupled to a bead 1009. The solution primer 1002 may bind to the circular template 1001 at the first adapter and/or second adapter. The bead-bound first stage concatemer 1003 and other bead-bound first stage concatemers generated from the template library may be deposited onto a substrate 1004 coated with a plurality of surface primers 1005. The surface primers may not be spaced apart in distinct clusters. The surface primers 1005 may bind to the sequence(s) of the first stage concatemers corresponding to the first adapter and/or second adapter. After deposition, the bead-bound first stage concatemers may be spaced apart from each other via the beads (e.g., 1009) acting as spacers. In some cases, the beads may self-assemble themselves as a layer on the substrate 1004. Effectively, each location of a bead-bound first stage concatemer may become an individually addressable location 1008. A single bead-bound first stage concatemer may be bound to multiple surface primers at the individually addressable location. In a second stage amplification, the bead-bound first stage concatemers (e.g., 1003) may be amplified on the substrate using the surface primers 1005, such as via MDA to generate second stage concatemers 1006 immobilized to the individually addressable locations 1008 of the substrate 1004. The beads may be washed away. It will be appreciated that the surface primers 1005 can include forward and reverse primers, and during second stage amplification, concatemers in the forward and reverse directions can be generated, such as via RCA and/or MDA. After the second stage amplification, an individually addressable location may have bound thereto a plurality of second stage concatemers, each of the second stage concatemers immobilized at a common individually addressable location comprising identical repeating oligonucleotide units. The second stage concatemers 1006 may then be sequenced on the substrate according to systems and methods described herein, such as by providing a plurality of sequencing primers 1007. The sequencing signals collected from each individually addressable location producing a signal may be attributed to a single distinct template insert. Where one type of concatemer has been sequenced first, the other type of concatemer may additionally be sequenced.

In FIG. 10E, circular templates (e.g., 1001) may be deposited onto a substrate 1004 patterned with individually addressable locations 1008. Each individually addressable location of a plurality of individually addressable locations on the substrate 1004 may comprise a cluster of surface primers 1005, the individually addressable locations discretely spaced apart from each other. After deposition, at most one circular template (e.g., 1001) may be bound to each individually addressable location. The circular templates may be amplified on the substrate using the surface primers 1005 such as via RCA and/or MDA to generate concatemers in the forward (e.g., 1003) and reverse (e.g., 1006) directions. It will be appreciated that at each individually addressable location, the cluster of surface primers 1005 includes forward and reverse primers. After amplification, an individually addressable location may have bound thereto a plurality of concatemers. One type of concatemer (forward or reverse) may then be sequenced on the substrate according to systems and methods described herein, such as by providing a plurality of sequencing primers 1007. The sequencing signals collected from each individually addressable location producing a signal may be attributed to a single distinct template insert. In some cases, the other type of concatemer may additionally be sequenced.

In some cases, the substrate 1004 may comprise clusters of surface primers 1005 comprising both forward primers and reverse primers, where the reverse primers are blocked for extension. After depositing the circular templates (e.g., 1001) on the substrate, the forward primers may be activated to amplify the circular templates to generate first stage concatemers (e.g., 1003). The first stage concatemers may be sequenced by adding a plurality of sequencing primers. Then, the reverse primers on the surface of the substrate 1004 may be unblocked and activated to amplify the first stage concatemers (e.g., 1003) to generate second stage concatemers (e.g., 1006). The second stage concatemers may be sequenced by adding a plurality of additional sequencing primers.

In FIG. 10F, circular templates (e.g., 1001) may be deposited onto a substrate 1004 coated with surface primers 1005. The surface primers may not be spaced apart in distinct clusters. After deposition, the circular templates may be amplified on the substrate using the surface primers 1005 such as via RCA and/or MDA to generate concatemers in the forward (e.g., 1003) and reverse (e.g., 1006) directions. It will be appreciated that surface primers 1005 include forward and reverse primers. After amplification, a plurality of concatemers may be bound to the substrate. One type of concatemer (forward or reverse) may then be sequenced on the substrate according to systems and methods described herein, such as by providing a plurality of sequencing primers 1007. The sequencing signals collected from each individually addressable location producing a signal may be attributed to a single distinct template insert. In some cases, the other type of concatemer may additionally be sequenced.

In some cases, the substrate 1004 may be coated with surface primers 1005 comprising both forward primers and reverse primers, where the reverse primers are blocked for extension. After depositing the circular templates (e.g., 1001) on the substrate, the forward primers may be activated to amplify the circular templates to generate first stage concatemers (e.g., 1003). The first stage concatemers may be sequenced by adding a plurality of sequencing primers. Then, the reverse primers on the surface of the substrate 1004 may be unblocked and activated to amplify the first stage concatemers (e.g., 1003) to generate second stage concatemers (e.g., 1006). The second stage concatemers may be sequenced by adding a plurality of additional sequencing primers.

In FIG. 10G, circular templates (e.g., 1001) may be bound to beads 1011 to form a bead assembly 1015 (bead-bound circular template). A bead may comprise a plurality of surface primers 1005 and click chemistry couplers 1012. One of the surface primers of the bead may bind to the circular template 1001 at the first adapter and/or second adapter to form the bead assembly. The bead assemblies may be deposited onto a substrate 1004 coated with complementary click chemistry couplers 1013. The click chemistry couplers 1012 of the beads 1011 may be configured to couple with the complementary click chemistry couplers 1013 via click chemistry pairings. After deposition, in some cases, the bead assemblies may be spaced apart from each other via the beads (e.g., 1011) acting as spacers. In some cases, the beads may self-assemble themselves as a layer on the substrate 1004. Effectively, each location of a bead assembly may become an individually addressable location. The bead assemblies (e.g., 1015) may be immobilized to the substrate 1004 by coupling the couplers 1012 and 1013. The circular templates may be amplified on the substrate using the surface primers 1005 on the bead (e.g., 1011) such as via RCA and/or MDA to generate concatemers in the forward (e.g., 1003) and reverse (e.g., 1006) directions. It will be appreciated that surface primers 1005 include both forward and reverse primers. After amplification, a plurality of concatemers may be immobilized to the substrate via the beads. One type of concatemer (forward or reverse) may then be sequenced on the substrate according to systems and methods described herein, such as by providing a plurality of sequencing primers 1007. The sequencing signals collected from each individually addressable location producing a signal may be attributed to a single distinct template insert. In some cases, the other type of concatemer may additionally be sequenced.

A click chemistry coupler and complementary click chemistry coupler pair may comprise functional groups configured to form covalent bonds upon reaction by click chemistry (e.g., Staudinger ligation or Diels-Alder chemistry) or by a click reaction. Coupling pairs are well known in the art. Examples of coupling pairs include, but are not limited to, biotin-avidin, carboxylic acid-amino group, NHS ester-amino group, maleimide-thiol, and Azide-DBCO (dibenzocyclooctyne).

In FIG. 10H, in a first stage amplification, a circular template 1001 may be amplified in solution using a solution primer 1002, such as via RCA to generate a first stage concatemer 1003. The solution primer 1002 may bind to the circular template 1001 at the first adapter and/or second adapter. The first stage concatemer 1003 and other first stage concatemers generated from the template library may be bound to dendrimers 1017, the dendrimers 1017 comprising a plurality of primers 1018 (e.g., in the dendrimer branches), to generate dendrimer assemblies 1019 (dendrimer-bound first stage concatemers). A single first stage concatemer may be bound to multiple primers at a dendrimer. In a second stage amplification, the first stage concatemers (e.g., 1003) bound to the dendrimers (e.g., 1017) may be amplified on the dendrimers using the primers 1018, such as via MDA to generate second stage concatemers 1006. It will be appreciated that the primers 1018 can include forward and reverse primers, and during second stage amplification, concatemers in the forward and reverse directions can be generated, such as via RCA and/or MDA. After amplification, a plurality of concatemers may be immobilized to the dendrimers. In some cases, the first stage concatemers (e.g., 1003) may be washed from the dendrimers. The dendrimer-bound second stage concatemers may be deposited onto a substrate 1004 patterned with individually addressable locations 1008, the individually addressable locations discretely spaced apart from each other. In some cases, each individually addressable location of a plurality of individually addressable locations on the substrate 1004 may comprise a surface chemistry (e.g., amination) that is different from a surface chemistry of non-individually addressable locations. Each individually addressable location of a plurality of individually addressable locations on the substrate 1004 may be configured to bind at most one dendrimer-bound second stage concatemer. Binders on individually addressable locations are described elsewhere herein. After deposition, at most one dendrimer-bound second stage concatemer can be immobilized to each individually addressable location. The second stage concatemers 1006 may then be sequenced on the substrate according to systems and methods described herein, such as by providing a plurality of sequencing primers 1007. The sequencing primers may be hybridized on or off the substrate 1004. The sequencing signals collected from each individually addressable location producing a signal may be attributed to a single distinct template insert.

In FIG. 10I, in a first stage amplification, a circular template 1001 may be amplified in solution using a solution primer 1002 coupled to a click chemistry coupler 1012, such as via RCA to generate a first stage concatemer 1003 coupled to the click chemistry coupler 1012. The solution primer 1002 may bind to the circular template 1001 at the first adapter and/or second adapter. The first stage concatemer 1003 and other first stage concatemers generated from the template library may be coupled to coupling beads 1021 to generate bead assemblies 1022 (bead-bound first stage concatemers). Each coupling bead may be coated with complementary click chemistry couplers 1013 which can react with the click chemistry couplers 1012 on the first stage concatemers 1003. A plurality of primers 1018 each coupled to a click chemistry coupler 1012 may be provided to the bead assemblies 1022. A single first stage concatemer may be bound to multiple primers of the plurality of primers 1018 at the bead. In a second stage amplification, the first stage concatemers (e.g., 1003) bound to the beads (e.g., 1021) may be amplified on the beads using the primers 1018, such as via MDA to generate second stage concatemers 1006. It will be appreciated that the primers 1018 can include forward and reverse primers, and during second stage amplification, concatemers in the forward and reverse directions can be generated, such as via RCA and/or MDA. The primers 1018 may be coupled to the beads (e.g., 1021) via the couplers 1012, 1013 prior to, during, or subsequent to the second stage amplification. A plurality of concatemers may be immobilized to the beads. The bead-bound second stage concatemers may be deposited onto a substrate 1004. The substrate may or may not be patterned with individually addressable locations, the individually addressable locations discretely spaced apart from each other. In some cases, where the substrate is not patterned, the bead-bound second stage concatemers may be spaced apart from each other via the beads (e.g., 1021) acting as spacers. In some cases, the beads may self-assemble themselves as a layer on the substrate 1004. Effectively, each location of a bead-bound second stage concatemer may become an individually addressable location on the substrate. After deposition, at most one bead-bound second stage concatemer can be immobilized to each individually addressable location. The second stage concatemers 1006 may then be sequenced on the substrate according to systems and methods described herein, such as by providing a plurality of sequencing primers 1007. The sequencing primers may be hybridized on or off the substrate 1004. The sequencing signals collected from each individually addressable location producing a signal may be attributed to a single distinct template insert.

In FIG. 10J, circular templates (e.g., 1001) may be bound to beads 1011 to form a bead assembly 1015 (bead-bound circular template). In some cases, linear templates 1031 may be bound to the beads 1011 first, and then circularized while bound to the bead. A bead (e.g., 1011) may comprise a plurality of surface primers 1005. One of the surface primers of the bead may bind to the circular template 1001 or the linear template 1031 at the first adapter and/or second adapter to form the bead assembly. The bead assemblies may be deposited onto a substrate 1004 patterned with a plurality of individually addressable locations 1008 which are discretely spaced apart from each other. In some cases, each individually addressable location of a plurality of individually addressable locations on the substrate 1004 may comprise a surface chemistry (e.g., amination) that is different from a surface chemistry of non-individually addressable locations. The circular templates may be amplified on the substrate using the surface primers 1005 on the bead (e.g., 1011) such as via RCA and/or MDA to generate concatemers in the forward (e.g., 1003) and reverse (e.g., 1006) directions. It will be appreciated that surface primers 1005 include forward and reverse primers. After amplification, a plurality of concatemers may be immobilized to the substrate via the beads. One type of concatemer (forward or reverse) may then be sequenced on the substrate according to systems and methods described herein, such as by providing a plurality of sequencing primers 1007. The sequencing signals collected from each individually addressable location producing a signal may be attributed to a single distinct template insert. In some cases, the other type of concatemer may additionally be sequenced.

In FIG. 10K, a circular template 1001 may be amplified in solution using a solution primer 1002, such as via RCA to generate concatemer 1003. The solution primer 1002 may bind to the circular template 1001 at the first adapter and/or second adapter. The concatemer 1003 and other concatemers generated from the template may be deposited onto a substrate 1004 patterned with a plurality of individually addressable locations 1008 which are discretely spaced apart from each other. In some cases, each individually addressable location of a plurality of individually addressable locations on the substrate 1004 may comprise a surface chemistry (e.g., amination, reactive moieties, etc.) that is different from a surface chemistry of non-individually addressable locations. In some cases, each individually addressable location may be configured to bind at most one concatemer. A concatemer may bind to an individually addressable location in a covalent manner (e.g., via click chemistry between reactive moieties on the concatemers and the individually addressable locations respectively, etc.) or non-covalent manner (e.g., electrostatic attraction, biotin-streptavidin interaction, etc.). The concatemers may then be sequenced on the substrate according to systems and methods described herein, such as by providing a plurality of sequencing primers 1007. It will be appreciated that the sequencing primers 1007 may be contacted and annealed to the concatemers (e.g., 1003) prior to, during, or subsequent to deposition of the concatemers on the substrate 1004, and primer extension reaction initiated (e.g., loading reagents such as polymerases and dNTPs) when they are ready to be sequenced. The sequencing signals collected from each individually addressable location producing a signal may be attributed to a single distinct template insert. In some cases, the sequencing product or extension product of such sequencing primers may additionally be sequenced by contacting and annealing the products with reverse primers.

In FIG. 10L, a circular template 1001 may be amplified in solution using a solution primer 1002, such as via RCA to generate concatemer 1003. The solution primer 1002 may bind to the circular template 1001 at the first adapter and/or second adapter. The concatemer 1003 and other concatemers generated from the template may be deposited onto a substrate 1004 which is unpatterned. In some cases, the substrate may comprise a surface chemistry (e.g., amination, reactive moieties, etc.) which can bind to the concatemers in a covalent manner (e.g., via click chemistry between reactive moieties on the concatemers and the individually addressable locations respectively, etc.) or non-covalent manner (e.g., electrostatic attraction, biotin-streptavidin interaction, hybridization to a primer, etc.). In the unpatterned surface chemistry, the surface may comprise moieties that are not spaced apart in uniformly distinct clusters. The concatemers may be immobilized to the substrate. The immobilized concatemers may then be sequenced on the substrate according to systems and methods described herein, such as by providing a plurality of sequencing primers 1007. It will be appreciated that the sequencing primers 1007 may be contacted and annealed to the concatemers (e.g., 1003) prior to, during, or subsequent to deposition of the concatemers on the substrate 1004, and primer extension reaction initiated (e.g., loading reagents such as polymerases and dNTPs) when they are ready to be sequenced. In some cases, the substrate may comprise a plurality of surface primers, and the concatemers may hybridize to the plurality of surface primers to immobilize the concatemers to the surface. The plurality of surface primers may then be used as sequencing primers and extended in a stepwise manner to sequence the concatemers. Each location producing a sequencing signal during one or more sequencing steps may be designated an individually addressable location. The sequencing signals collected from an individually addressable location may be attributed to a single distinct template insert. In some cases, the sequencing product or extension product of such sequencing primers may additionally be sequenced by contacting and annealing the products with reverse primers.

In a variation of this and other workflows (not illustrated), the concatemer 1003 may be deposited onto a surface comprising blocked oligonucleotides (e.g., which are complementary to the solution primer 1002). For example, the blocked oligonucleotides may be blocked with a dideoxynucleotide (e.g., ddNTP). The blocked oligonucleotides may be attached to the substrate surface and immobilize the concatemers (e.g., 1003) onto the surface via hybridization. On the substrate, sequencing primers 1007 may be provided to the concatemers hybridized to the blocked oligonucleotides to initiate a first sequencing reaction.

In FIG. 10M, an adapter-ligated linear template 1014 may be circularized and amplified on surface, as described in the circularization workflows with respect to FIGS. 9B-9C. In some cases, adapter-ligated linear templates (e.g., 1014) may be deposited onto a substrate 1004 comprising a plurality of surface primers 1015. The substrate may be patterned with the surface primers 1015 such that each surface primer is discretely spaced apart from other surface primers (as illustrated in FIG. 10M), or a cluster of the surface primers are discretely spaced apart from other clusters (not illustrated). The substrate may be unpatterned wherein the surface primers are not discretely spaced apart from each other. The surface primers may be arranged in a pattern or randomly. A surface primer may act as a splint molecule which attaches to the two ends of an adapter-ligated linear template to circularize the template molecule. The two ends may be ligated to generate a circularized template molecule 1001. In other cases, circularized template molecules (e.g., 1001) may be generated off the substrate and deposited onto the substrate 1004 comprising the plurality of surface primers 1015. In either case, circularized template molecules (e.g., 1001) may be annealed to the surface primers on the substrate 1004. The circular templates may be amplified on the substrate using the surface primers 1015 such as via RCA to generate concatemers (e.g., 1003) immobilized to the substrate. The immobilized concatemers may then be sequenced on the substrate according to systems and methods described herein, such as by providing a plurality of sequencing primers 1007. Each location producing a sequencing signal during one or more sequencing steps may be designated an individually addressable location. The sequencing signals collected from an individually addressable location may be attributed to a single distinct template insert. In some cases, the sequencing product or extension product of such sequencing primers may additionally be sequenced by contacting and annealing the products with additional sequencing primers.

In some cases, the substrate 1004 may comprise an additional plurality of primers such that the substrate comprises both forward and reverse surface primers. The reverse surface primers may be used to bind to and amplify the first stage concatemers (products of RCA on surface) to generate second stage concatemers, such as via MDA. It will be appreciated that because the surface primers include both forward and reverse primers, during second stage amplification, concatemers in the forward and reverse directions can be generated, such as via RCA and/or MDA. The second stage concatemers may then be sequenced on the substrate according to systems and methods described herein, such as by providing a plurality of additional sequencing primers. The sequencing signals collected from a distinct individually addressable location producing a signal may be attributed to a single distinct template insert. Where one type of concatemer has been sequenced first, the other type of concatemer may additionally be sequenced.

FIG. 10N illustrates a circular template 1001 annealed to a primer 1002 bound to a reactive moiety 1016a (“X”). Alternatively, an adapter-ligated linear template (e.g., 1014) may be circularized and the two ends ligated by using the primer 1002, bound to reactive moiety 1016a, as a splint molecule (not illustrated). Primer-annealed circular template assemblies may be deposited onto a substrate 1004 comprising a plurality of additional reactive moieties 1016b (“Y”) that are configured to react and/or conjugate to the reactive moiety 1016a to bind the primer-annealed circular template assemblies to the surface. The substrate may be patterned with the additional reactive moieties 1016b such that each additional reactive moiety is discretely spaced apart from other additional reactive moieties (as illustrated in FIG. 10N), or a cluster of the additional reactive moieties are discretely spaced apart from other clusters (not illustrated). The substrate may be unpatterned wherein the additional reactive moieties are not discretely spaced apart from each other. The additional reactive moieties may be arranged in a pattern or randomly. Once the circularized template molecules (e.g., 1001) are immobilized to the surface, the circular templates may be amplified on the substrate using the primers 1002 such as via RCA to generate concatemers (e.g., 1003) immobilized to the substrate. The immobilized concatemers may then be sequenced on the substrate according to systems and methods described herein, such as by providing a plurality of sequencing primers. Each location producing a sequencing signal during one or more sequencing steps may be designated an individually addressable location. The sequencing signals collected from an individually addressable location may be attributed to a single distinct template insert. In some cases, the sequencing product or extension product of such sequencing primers may additionally be sequenced by contacting and annealing the products with additional sequencing primers.

The reactive moiety and additional reactive moieties may be any coupling pair described herein, such as biotin-avidin, carboxylic acid-amino group, NHS ester-amino group, maleimide-thiol, and Azide-DBCO pairs. In some cases, the reactive moiety and/or the additional reactive moiety may be a single group unit. In some cases, the reactive moiety and/or the additional reactive moiety may be a multi-group unit. Beneficially, the use of reactive moiety pairs obviates the need to control the primer density of the surface primers on the substrate when preparing the substrate. In workflows that use substrates comprising surface primers, such as the workflow described with respect to FIG. 10M, the density of the surface primers may be optimized and controlled such that there is a sufficient number of primers to bind to a large number of nanoballs but not too many primers to inhibit the circular templates from annealing to a primer. Further, a substrate comprising reactive moieties on its surface may be stored in more lenient conditions than a substrate comprising primer molecules on its surface. Overall, the workflow may be more cost-efficient as when circular templates are deposited to a surface primer-coated surface, some of the surface primers may be unoccupied and wasted. Pre-annealing the primer to the circular template in a tube at relatively higher temperatures may also ensure specificity and overcome secondary structures (e.g., high GC templates).

It will be appreciated that various modifications can be made to any of the workflows described above. For example, in any of these workflows, the substrate may be patterned or unpatterned. The sample loaded onto the substrate may be in the form of a linear template, a circular template, a concatemer (e.g., nanoball), any of the above with or without beads, or a combination thereof. The sample may be immobilized to the substrate via hybridization to a surface primer on the surface, via coupling of coupling pairs on the surface and the sample, respectively, via covalent binding, via non-covalent binding, via electrostatic attraction, via an affinity of the sample to the surface, etc. For example, the surface of the substrate may comprise a single type of surface primer, two types of surface primers (e.g., forward and reverse primers), more than three types of surface primers, no surface primers, amination (e.g., aminosilane), etc. First stage amplification (e.g., RCA) may be performed on or off the surface of the substrate to generate first stage concatemers. In some cases, only one stage of amplification may be performed. In some cases, multiple stages (e.g., two or more) of amplification may be performed. Sequencing primers may be pre-hybridized to a concatemer off the substrate, and the concatemer-sequencing primer assembly loaded during sample loading. Sequencing primers may be hybridized to a concatemer after the concatemer is loaded and immobilized to the substrate. The first stage concatemer products may or may not be sequenced by providing or activating sequencing primers to the concatemers. Second stage amplification (e.g., MDA, RCA, etc.) may or may not be performed using the first stage concatemers to generate second stage concatemer products. The second stage concatemer products may or may not be sequenced by providing or activating additional sequencing primers to the concatemers. At least one of the stages of the concatemer products may be sequenced while immobilized to the substrate. In some cases, both first stage and second stage concatemers may be sequenced while immobilized to the substrate. In some cases, sequencing reads in a single direction (e.g., forward or reverse) may be generated from the substrate. In some cases, sequencing reads in both directions (e.g., forward and reverse) may be generated from the substrate, for example to generate paired end reads. A distinct location from which one or more sequencing signals are collected may be designated an individually addressable location, and all signals collected from an individually addressable location may be attributed to a single template insert, for example generated from sequencing the template insert or derivatives thereof (e.g., concatemer amplification product of the template insert).

In another example, in the workflow of FIG. 10J, alternatively, the substrate may be un-patterned, and upon deposition of the bead-bound concatemers, the bead-bound concatemers may be spaced apart from each other via the beads (e.g., 1011) acting as spacers. In some cases, the beads may self-assemble themselves as a layer on the substrate 1004. Effectively, each location of a bead-bound second stage concatemer may become an individually addressable location on the substrate. In some cases, for workflows where a certain stage of amplification (e.g., RCA, MDA) is described to be performed in solution, the amplification stage may be further broken up into different sub-stages; for example, a first round of amplification may be performed in solution to produce relatively short concatemers, and a second round of amplification may be performed after deposition on the substrate.

In some cases, ethylene carbonate may be added during amplification to aid in primer annealing. For example, ethylene carbonate may be added for in solution amplification, e.g., in solution rolling circle amplification (RCA) or for on surface RCA. Alternatively, or in addition, ethylene carbonate may be added during sequencing primer hybridization on the surface. Beneficially, ethylene carbonate may reduce secondary structures during rolling circle amplification, increasing the length of concatemers, which by increasing the number of repeating units increases sequencing signal. Furthermore, ethylene carbonate may also increase primer hybridization efficiency, which also increases sequencing signal. Use of ethylene carbonate for sequencing on a surface is described in International Patent Pub. No. 2023/141430A1, which is entirely incorporated herein by reference for all purposes. The further benefits of using ethylene carbonate are demonstrated in Examples 2-3.

In some cases, the ethylene carbonate may be dissolved in water to form a solution comprising a specific concentration of ethylene carbonate. In some embodiments, the ethylene carbonate may be dissolved in a buffer. In some embodiments, the buffer may be a tris-based aqueous solution. In some cases, the ethylene carbonate can be provided at a concentration of about, at least about, and/or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or greater.

In some cases, reagents such as magnesium ions, ethylenediaminetetraacetic acid (EDTA), isopropyl alcohol (IPA), PEG, albumin (e.g., Bovine serum albumin (BSA)), ethylene carbonate, and other reagents, or a combination thereof may be added to the surface comprising the concatemers to stabilize the concatemers, such as to help reduce shearing and compact nanoball structures.

In some cases, after amplification (e.g., RCA), the output mixture may be enriched for successfully concatemerized samples, such as by contacting the output mixture with an enzyme that specifically digests or otherwise degrades or removes single-stranded DNA, thereby leaving only double-stranded DNA (e.g., successful nanoballs). Example enzymes include exonuclease I (ExoI), exonuclease T (ExoT), exonuclease VII (ExoVII), and Mung Bean nuclease (nuclease MB).

### Surface Conditions

A substrate that immobilizes concatemers may comprise various surface conditions, for example, various surface chemistry.

In some cases, the surface chemistry may comprise an amine. In some cases, the surface chemistry may comprise a silane, such as an aminosilane or azidosilane. In some cases, the surface chemistry may comprise hexamethyldisilazane (HMDS) or (3-aminopropyl)triethoxysilane (APTMS). In some cases, the surface chemistry may comprise 3-(azido propyl)triethoxysilane or azido-PEG triethoxysilane (e.g., aizdo-PEG5-triethoxysilane). In some cases, the surface chemistry may comprise a spacer or multiple types of spacers. The spacer may comprise polyethylene glycol (PEG). In some cases, the surface chemistry may comprise a surface primer molecule or any oligonucleotide molecule that has any degree of affinity towards another molecule. Beneficially, aminosilane surfaces may carry a positive charge which has an affinity towards the generally negative charges carried by nucleic acid molecules. Beneficially azidosilane surfaces may permit flexibility of modifying the surface with various molecules or moieties via click reaction between the azide moieties on the surface and DBCO moieties that may be conjugated to the various molecules or moieties. Beneficially, PEG spacers may increase hydrophilicity of the surface and increase the binding efficiency of circular templates or concatemers (e.g., nanoballs) to the surface. Beneficially, a surface primer molecule or any oligonucleotide molecule may be used directly as amplification primers or sequencing primers. The substrate may comprise any one or any combination of the above surface chemistries.

A substrate may be patterned with any or multiple of the above surface chemistries. For example, the surface may comprise a distinct location that comprises a first type of surface chemistry that is differentiated from a second location that lacks the first type of surface chemistry and/or comprises a second different type of surface chemistry. For example, the patterns of locations may be created with lithographic techniques. In one example, the substrate is patterned, comprising a plurality of APTMS locations, which are separated by HMDS locations. In another example, the substrate is patterned, comprising a plurality of clusters of surface primer locations, which are separated by locations that do not have surface primers.

A substrate may be unpatterned but comprise any or multiple of the above surface chemistries. For example, the surface may comprise moieties that are not spaced apart in uniformly distinct clusters. Any part of the surface may indiscriminately bind to a nucleic acid molecule. In one example, the substrate is unpatterned, comprising an aminosilane (e.g., APTMS) or azidosilane. In another example, the substrate is unpatterned, comprising PEG spacers on an azidosilane surface, the PEG spacers bound to the azidosilane surface via DBCO-azide click reactions (PEG-DBCO molecules are clicked with azides on the surface). In another example, the substrate is unpatterned, comprising surface primer molecules or other oligonucleotide molecules. In another example, the substrate is unpatterned, comprising surface primer molecules or other oligonucleotide molecules interspersed with PEG spacers (e.g., such that the PEG spacers block non-primer surfaces on the substrate).

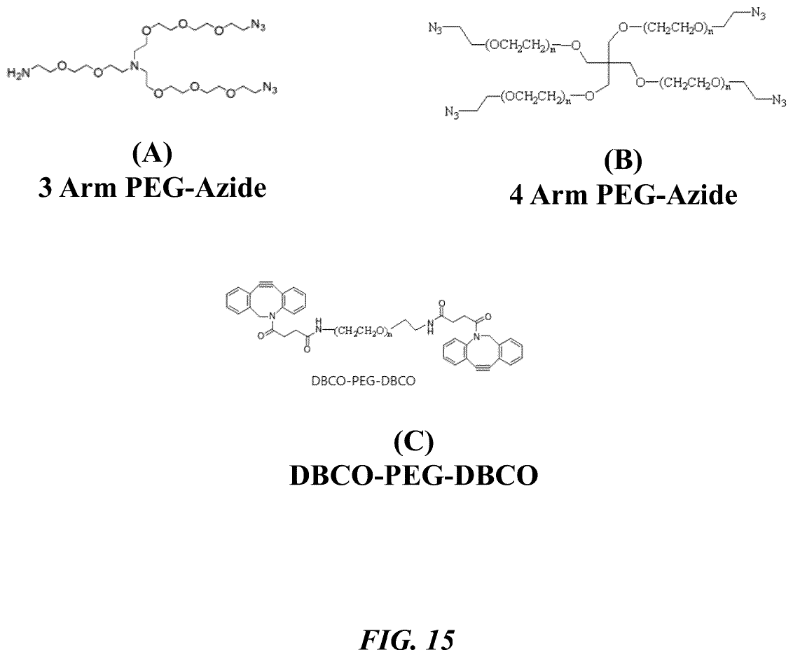

In some cases, surface primer molecules may be attached to a substrate surface via click chemistry reaction, such as a DBCO-azide click chemistry reaction. It will be appreciated that alternative click chemistry reactions can also be used. It will be appreciated that while click chemistry reactions are used in these examples, alternative conjugation chemistries can also be used. In an example, a DBCO-conjugated surface primer molecule may be clicked to an azide moiety on an azidosilane surface. In another example, a DBCO-PEG-DBCO spacer molecule may be clicked to an azide moiety on an azidosilane surface, and an azide-conjugated surface primer molecule may be clicked to the DBCO moiety on the DBCO-PEG DBCO spacer molecule. In another example, a DBCO-PEG-DBCO spacer molecule may be clicked to an azide moiety on an azidosilane surface, a second spacer molecule comprising a azide-multi-arm PEG may be clicked to a DBCO moiety on the DBCO-PEG DBCO spacer molecule, and a DBCO-conjugated surface primer molecule may be clicked to an azide moiety of the azide-multi-arm PEG second spacer molecule. In another example, a DBCO-PEG-DBCO spacer molecule may be clicked to an azide moiety on an azidosilane surface, a second spacer molecule comprising a azide-PEG-azide may be clicked to a DBCO moiety on the DBCO-PEG DBCO spacer molecule, and a DBCO-conjugated surface primer molecule may be clicked to the azide moiety of the azide-PEG-azide second spacer molecule. FIG. 15 illustrates in panel (A) an example 3 Arm PEG-Azide molecule, in panel (B) an example 4 Arm PEG-Azide molecule, and in panel (C) an example DBCO-PEG-DBCO molecule. As used herein, the notation azide- or -azide may refer to an azide-conjugated moiety and the notation DBCO- or -DBCO may refer to a DBCO-conjugated moiety. It will be appreciated that where a DBCO-azide reaction is depicted, an azide can be substituted for the DBCO and the DBCO substituted for the azide.

As such, a substrate may comprise one or more layers. For example, the final surface chemistry may be prepared in multiple layers. In some cases, only one of the layers may be exposed at the surface of the substrate, e.g., exposed to the open atmosphere above the substrate surface and/or available to contact a nucleic acid sample deposited on the surface. In some cases, at least a portion of each of the layers may be exposed at the surface of the substrate, e.g., exposed to the open atmosphere above the substrate surface and/or available to contact a nucleic acid sample deposited on the surface. In some cases, at least a portion of some but not all of the layers may be exposed at the surface of the substrate, e.g., exposed to the open atmosphere above the substrate surface and/or available to contact a nucleic acid sample deposited on the surface.

In some cases, two adjacent layers may be coupled together via click chemistry reaction(s) between coupling pairs, as described above (e.g., a PEG layer coupled to an azide layer on the surface, an oligonucleotide layer coupled to a PEG layer coupled to an azide layer on the surface, etc.). It will be appreciated that alternative conjugation chemistries can also be used to couple layers. In some cases, a layer may be deposited onto another layer, such as via chemical vapor deposition (CVD). In some cases, various lithography, methods may be used to generate, remove, and/or modify a layer, such as photolithography methods, nanoimprint lithography (NIL), extended ultraviolet lithography (EUV), electron beam lithography, or optical lithography methods such as i-line stepper/scanner lithography, deep ultraviolet (DUV) lithography, extreme ultraviolet (EUV) lithography, and X-ray lithography. The substrate may comprise any of the substrate materials described elsewhere herein. In some cases, one or more of the layers may comprise SiO₂, SiN, glass, fused silica, metal oxide, or other oxide-based materials. In some cases, a layer may comprise a self-assembled monolayer (SAM) coating.

A PEG spacer may be provided at any useful length, with any number of repeating units (e.g., ethylene oxide). For example, the PEG may have at least about, at most about, and/or about 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400 or more repeating units. In some cases, the PEG may be provided at any useful molar mass, for example, at least about, at most about, and/or about 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10 k, 10.5 k, 11 k, 11.5 k, 12 k, 12.5 k, 13 k, 13.5 k, 14 k, 14.5 k, 15 k, 15.5 k, 16 k, 16.5 k 17 k, 17.5 k, 18 k, 18.5 k, 19 k, 19.5 k, 20 k or more g/mol or daltons (Da). The PEG may be a linear PEG or multi-arm PEG (e.g., 3-arm PEG, 4-arm PEG, 5-arm PEG, 6-arm PEG, 7-arm PEG, 8-arm PEG, etc.). The PEG may comprise a methyl terminated PEG.

In some cases, where the surface comprises an azide layer and comprises excess azide groups (e.g., after applying a PEG layer by click coupling to a subset of the azide groups), at least a subset of the excess azide groups may be converted to amines which are positively charged to increase an affinity for nucleic acid samples. The —N₃ azide group may be reduced to a —NH₂ amine group via contacting a reducing agent such as DTT, TCEP, etc. The presence of amine groups on the surface or on a surface comprising an amine layer may help stabilize the binding and/or 3D structure of a concatemer (e.g., nanoball) on the surface, such as by generally flattening the structure of the concatemer towards the surface. A reducing agent may be applied to an azide-containing surface prior to, during, or subsequent to loading concatemers on the surface to generate positively charged amine groups on the surface. A reducing agent may be applied to an azide-containing surface prior to, during, or subsequent to generating concatemers on the surface to generate positively charged amine groups on the surface.

### Sequencing Methods

Provided herein are systems, methods, compositions, and kits for sequencing, including paired end sequencing, of concatemers.

During sequencing by synthesis, a sequencing primer may be hybridized to a template (e.g., to a primer binding site on the template) and extended in a stepwise manner by, in each extension step, contacting the hybrid with nucleotide reagents of known canonical base type(s). The extended or extending sequencing primer may also be referred to herein as a growing strand. An extension step may be a bright step (also referred to herein, in some cases, as labeled step or detected step) or a dark step (also referred to herein, in some cases, as an unlabeled step or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted.

Flow-based sequencing methods and non-terminated sequencing-by-synthesis methods have been generally described elsewhere herein. In terminated sequencing-by-synthesis methods, a bright step may comprise terminated nucleotides (e.g., reversibly terminated nucleotides). In some cases, a bright step may comprise a single nucleotide base type or a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides (e.g., and not unterminated nucleotides) a single nucleotide base may be incorporated into a growing strand. In an extension step comprising a mixture of reversibly terminated and unterminated nucleotides, more than one nucleotide base may be incorporated into a growing strand. Sequencing methods of the present disclosure may comprise flow-based sequencing, non-terminated sequencing, and/or terminated sequencing.

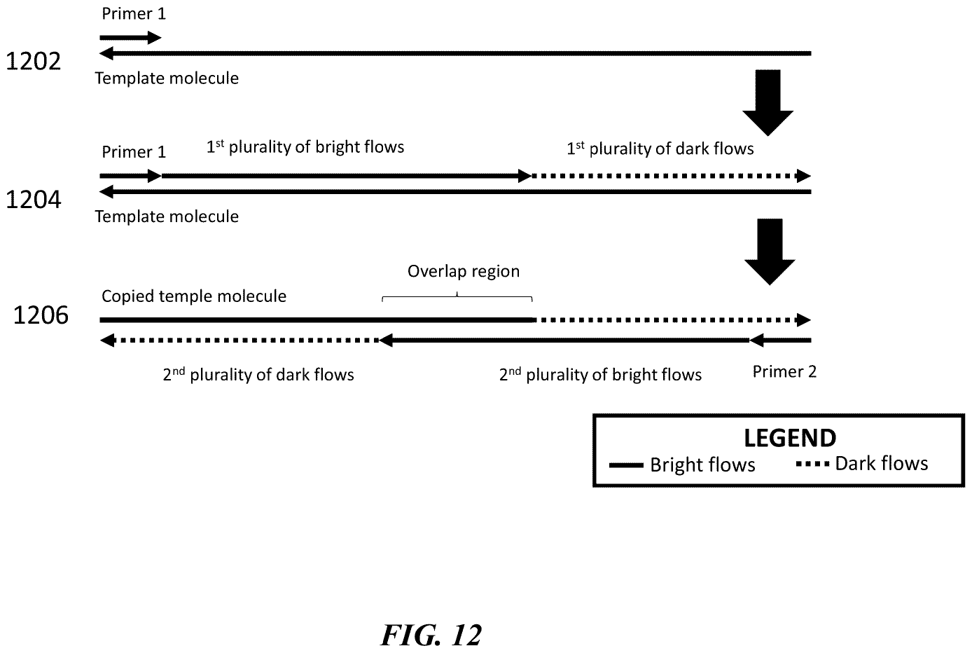

FIG. 12 illustrates a schema for paired end sequencing comprising multiple sequencing runs. Either or both sequencing runs may use non-terminated nucleotides, terminated nucleotides, or a mixture of both.

In step 1202, a first sequencing primer is annealed (e.g., is hybridized) to a template molecule at a first primer binding site. The template molecule may be any of the concatemers immobilized to the surface described elsewhere herein. For a first number of sequencing flows (e.g., a number of bright flows), or for a first region of the template molecule, labeled nucleotides are added and are incorporated into the extending first primer. In each of the first number of sequencing flows, each incorporated nucleotide may be detected. After detection, the labeling moiety and/or the terminating moiety may be removed (e.g., cleaved) from the incorporated nucleotide.

At least a subset or all of the nucleotides added during bright flows may be labeled. The nucleotides added in each bright flow comprise four, three, two, or one canonical base types. The nucleotides may be reversibly terminated or non-terminated. In some cases, the nucleotides of each base type may comprise a respective label moiety. In some cases, each respective label moiety may be a different fluorescent label (e.g., fluorescent moieties with different excitation/emission spectra). In some cases, nucleotides of one of the added base types are unlabeled. In some cases, for reversibly terminated sequencing, each respective label moiety may comprise a different number of the same fluorescent label (e.g., where a first base type is labeled with one fluorescent moiety of a first type and a second base type is labeled with three fluorescent moieties of the same type).

In step 1204, for a second number of sequencing flows (e.g., a number of dark flows), or for a second region of the template molecule, nucleotides are added and are incorporated into the extending first primer. In the second number of sequencing flows, at most only a subset of incorporated nucleotides is detected or no incorporation is detected. In some cases, nucleotides added in the second number of sequencing flows are labeled and unterminated. In some cases, at least some of the nucleotides in the second number of sequencing flows are unlabeled and unterminated. In some cases, at least some of the nucleotides in the second number of sequencing flows are terminated. The dark flows may comprise nucleotides of one, two, three, or four canonical base types. In some cases, nucleotides of one of the added base types are reversibly terminated. In some cases, nucleotides of one or more of the added base types are labeled. In some cases, nucleotides added in dark flows may be unlabeled.

The extended first sequencing primer comprises a copied template molecule (e.g., a molecule that is a reverse complement to the template molecule). After the second number of sequencing flows (e.g., the dark flows), the copied template molecule and the template molecule are denatured (e.g., exposed to conditions sufficient to denature the copied template molecule from the template molecule). The copied template molecule may be a concatemer. The copied template molecule may be immobilized to the substrate surface. For example, in 1202, the first sequencing primer may be conjugated to the surface, and the template molecule (e.g., concatemer) may be annealed to the first sequencing primer such that the extended molecule, the copied template molecule is conjugated to the surface and upon denaturation the template molecule is washed away. In some cases, the substrate may comprise a second sequencing primer and the copied template molecule, upon denaturation, may anneal to the second sequencing primer, thus immobilizing the copied template molecule to the substrate.

In step 1206, a second sequencing primer is annealed (e.g., is hybridized) to a second sequencing primer binding site in the copied template molecule. The second sequencing primer is extended along the copied template molecule via a first plurality of bright flows followed by a plurality of dark flows. For a first number of sequencing flows (e.g., a number of bright flows), or for a first region of the copied template molecule, labeled nucleotides are added and are incorporated into the extending second primer (e.g., nucleotides comprising a labeling moiety and a reversibly terminating moiety). In each of the first number of sequencing flows, each incorporated nucleotide may be detected. After detection, the labeling moiety and/or the terminating moiety is removed (e.g., cleaved) from the incorporated nucleotide. For a second number of sequencing flows (e.g., a number of dark flows), or for a second region of the template molecule, nucleotides are added and are incorporated into the extending second primer. In the second number of sequencing flows, at most only a subset of incorporated nucleotides is detected or no incorporation is detected. That is, detection steps may be performed every n flows, where n is an integer greater than 1. N may be 2, 3, 4, 5, 6, 7, 8, 9, 10, etc. In some cases, nucleotides added in the second number of sequencing flows are unterminated. In some cases, at least some of the nucleotides in the second number of sequencing flows are unlabeled. The dark flows may comprise nucleotides of one, two, three, or four canonical base types. In some cases, nucleotides of one or more of the added base types are reversibly terminated. In some cases, nucleotides of one or more of the added base types are labeled.

The first sequencing primer binding site is located at or adjacent to the 3′ end of the template molecule, and the second sequencing primer binding site is located at or adjacent to the 3′ end of the copied template molecule. In some cases, there will be an overlap in loci covered by bright flows in the template molecule and the copied template molecule (see ‘Overlap region’ in step 1206). Such loci that have been sequenced twice with bright flows will have decreased base call error rates than loci that were only sequenced once with bright flows. Further, as sequencing progresses along a length of a template, the sequencing quality may decrease due to phasing problems (e.g., leading and lagging signals due to misincorporation and/or uncompleted extension reactions during each extension steps)—beneficially, this method permits collection of higher quality signals corresponding to both ends of the template molecule, one from each of the template and copied template molecules.

In some cases, a method for paired end sequencing may comprise, for first strand sequencing, extending a first sequencing primer annealed to a first primer binding site in the template concatemer via bright steps (e.g., with detection) until the signal quality drops below a predetermined threshold, and then continuing to extend via dark steps (e.g., without detection) with a strand displacing polymerase. Because the template is a concatemer, multiple first sequencing primers may be simultaneously extended from multiple first primer binding sites in the concatemer, and one extending primer molecule may eventually displace another extending primer molecule from the concatemer as the extension steps progress. The dark step extensions may be terminated, such as by incorporating a ddNTP. The extended products, which each comprises a reverse complement of the template, may comprise a reverse primer binding site. The method may further comprise, for second strand sequencing, annealing a second sequencing primer to the reverse primer binding site and extending the second sequencing primer via bright steps (e.g., with detection). The sequencing reads generated from the first and second strands may be processed as paired end reads.

In some cases, a method for paired end sequencing may comprise, for first strand sequencing, extending a first sequencing primer annealed to a first primer binding site in the template concatemer via bright steps (e.g., with detection) until the signal quality drops. A strand displacement primer may then be annealed to a second primer binding site on the template concatemer, and extended via dark steps (e.g., without detection) with a strand displacing polymerase. Because the template is a concatemer, multiple primers may be simultaneously extended from multiple primer binding sites in the concatemer, and one extending primer molecule may eventually displace another extending primer molecule from the concatemer as the extension steps progress. The dark step extensions may be terminated, such as by incorporating a ddNTP. The extended products, which each comprises a reverse complement of the template, may comprise a reverse primer binding site. The method may further comprise, for second strand sequencing, annealing a second sequencing primer to the reverse primer binding site and extending the second sequencing primer via bright steps (e.g., with detection). The sequencing reads generated from the first and second strands may be processed as paired end reads. Addition of the strand displacement primer may accelerate the kinetics. In some cases, the reverse primer binding site may be the reverse complement of one of the first primer binding site and the second primer binding site.

In some cases, a method for paired end sequencing may comprise, per the workflow of FIG. 10C, for first strand sequencing, extending a first sequencing primer annealed to a first primer binding site in the template concatemer via bright steps (e.g., with detection). Then, a strand displacement primer may be annealed to a second primer binding site on the template concatemer, and extended via dark steps (e.g., without detection) with a strand displacing polymerase. Because the template is a concatemer, multiple primers may be simultaneously extended from multiple primer binding sites in the concatemer, and one extending primer molecule may eventually displace another extending primer molecule from the concatemer as the extension steps progress. The dark step extensions may be terminated, such as by incorporating a ddNTP. The extended products, which each comprises a reverse complement of the template, may comprise a reverse primer binding site. The method may further comprise, for second strand sequencing, annealing a second sequencing primer to the reverse primer binding site and extending the second sequencing primer via bright steps (e.g., with detection). The sequencing reads generated from the first and second strands may be processed as paired end reads. Addition of the strand displacement primer may accelerate the kinetics. In some cases, the reverse primer binding site may be the reverse complement of one of the first primer binding site and the second primer binding site.

A sequencing by synthesis method, which may or may not be paired end, may comprise any number of bright steps and any number of dark steps. A sequencing by synthesis method may comprise any number of bright regions (consecutive bright steps) and any number of dark regions (consecutive dark steps). In some cases, the dark steps or dark regions may be used to accelerate or fast forward through certain regions of the template during sequencing. In some cases, the dark steps or dark regions may be advantageous to correct phasing problems.

In some cases, a method for sequencing may comprise sequencing a same template strand multiple times to generate robust sequencing data (e.g., a high quality sequencing read) corresponding to the template strand. In some cases, a method for sequencing may comprise sequencing a same template strand multiple times and sequencing a same reverse complement strand of the template strand multiple times (e.g., both forward and reverse strands) to generate robust sequencing data (e.g., a high quality paired end read) corresponding to the template strand. A method for re-sequencing a template strand (which may be a forward strand or reverse strand) may comprise annealing a first sequencing primer to the template strand, extending the first sequencing primer through at least a first portion of the template strand via any combination of bright steps and/or dark steps to generate first sequencing data, denaturing the extended strand from the template strand, annealing a second sequencing primer to the template strand, and extending the second sequencing primer through at least a second portion of the template strand via any combination of bright steps and/or dark steps to generate second sequencing data, and processing (e.g., combining, comparing, matching, aligning, resolving, etc.) the first sequencing data and the second sequencing data to generate a sequencing read of the template strand. A template strand may be denatured and re-sequenced any number of times, such as about, at least about, and/or at most about 1, 2, 3, 4, 5, 6, 7, 8, 9, 10 or more times, such as by annealing an nth sequencing primer to the template strand, and extending the nth sequencing primer through at least an nth portion of the template strand. The different n sequencing primers may comprise the same or different sequences, which may bind to same or different primer binding sites on the template strand, respectively. The different nth portions on the template strand may refer to the same portions or different portions on the template strand. Two portions may be partially overlapping, completely overlapping (for one or both portions), or non-overlapping. The respective extensions through the template strand in the different sequencing runs may use the same or different nucleotide reagents (e.g., non-terminated nucleotides during a first sequencing run, terminated during a second sequencing run; green dye-labeled nucleotides during a first sequencing run, red dye-labeled nucleotides during a second sequencing run; labeled A-, T-, G-bases and unlabeled C-base nucleotides during a first sequencing run, labeled A-, T-, C-bases and unlabeled G-base nucleotides during a second sequencing run; 5% labeled A bases during a first sequencing run; 100% labeled A bases during a second sequencing run; etc.). The respective extensions through the template strand in the different sequencing runs may have the same flow order or flow cycle of nucleotide reagents. The respective extensions through the template strand in the different sequencing runs may have different flow orders or flow cycles of nucleotide reagents (e.g., A->T->G->C single base flow cycle order during a first sequencing run, T->A->G->C single base flow cycle order during a second sequencing run; A/T/G/C 4-base flow cycle order during a first sequencing run; A/T/G->A/T/C 3-base flow cycle order during a second sequencing run, etc.). Denaturing may comprise contacting the double-stranded nucleic acid molecule with denaturing agents, such as sodium hydroxide (NaOH) or ethylene carbonate. An entire substrate may be subjected to resequencing by, after a first sequencing run, contacting the entire surface with a solution comprising a denaturing agent, contacting the entire surface with a solution comprising sequencing primers under conditions sufficient to anneal them to template nucleic acid strands immobilized to the substrate, and subjecting them to extension reactions.

The systems and methods for processing open substrates described elsewhere herein may be used for any of the operations described in these workflows, such as sample loading, circularization, amplification, sequencing, and/or data processing operations. For example, the sample may be loaded to a substrate in a sample loading station or a processing station. In some cases, the sample may be subjected to amplification conditions in the sample loading station and/or the processing station. The substrate may be rotated prior to, during, or subsequent to sample loading, circularization, amplification, and/or sequencing operations. The substrate may be placed within sample processing environments (e.g., enclosed by chamber and lid) during sequencing steps, such as travelling between processing and detection stations.

A method for sequencing concatemers may comprise providing a substrate comprising a plurality of concatemers immobilized thereto, extending sequencing primers hybridized to the plurality of concatemers in a series of extension steps, and detecting signals, or lack thereof, from the substrate during or subsequent to at least a subset of steps or each step of the series of extending steps to determine sequencing reads corresponding to the plurality of concatemers. The extending of the sequencing primers may comprise providing, in each extension step, a plurality of nucleotides, of which at least a subset of nucleotides is labeled, under conditions sufficient to incorporate the plurality of nucleotides if they are complementary to the next bases in the respective templates of the extending sequencing primers (e.g., the concatemers). For example, at least and/or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the plurality of nucleotides may be labeled. In some cases, a single canonical base type (e.g., A, T/U, G, or C), two base types, three base types, or four base types may be provided during a single extension step. A single canonical base type may be labeled with a single dye type. Where two or more base types are provided, the different base types may be labeled by the same dye type or different dye types. The nucleotides may be reversibly terminated. The nucleotides may be non-terminated. The substrate may be patterned. The substrate may be unpatterned. The method may further comprise rotating the substrate prior to, during, or subsequent to providing of the plurality of nucleotides during the extension steps. The method may further comprise rotating the substrate prior to, during, or subsequent to detecting of the signals, or lack thereof.

The method may further comprise loading the plurality of concatemers on the substrate to immobilize the plurality of concatemers on the substrate. The substrate may be absent of surface primers. The substrate may comprise surface primers which may be used as the sequencing primers after hybridizing to the plurality of concatemers. The method may further comprise amplifying a plurality of circular templates, off the substrate and in solution, to generate the plurality of concatemers. The amplifying may comprise RCA. The plurality of circular templates may comprise different template inserts. The method may further comprise circularizing a plurality of linear templates comprising different template inserts using splint molecules and ligating the respective two ends of the plurality of linear templates to generate the circular templates. In some cases, the splint molecules may be removed from the circular templates prior to amplifying the plurality of circular templates. In some cases, the splint molecules may be used as primers for the amplification of the plurality of circular templates.

The method may further comprise loading a plurality of circular templates on the substrate, wherein the substrate comprises a plurality of surface primers, to hybridize the plurality of circular templates to the plurality of surface primers, and using the plurality of surface primers to amplify the plurality of circular templates to generate the plurality of concatemers immobilized to the substrate. Alternatively, the method may further comprise loading a plurality of circular templates on the substrate, wherein the plurality of circular templates are hybridized to a plurality of primers that are conjugated to reactive moieties, and wherein the substrate comprises additional reactive moieties configured to couple to the reactive moieties, to bind the plurality of circular templates to the substrate, and using the plurality of primers to amplify the plurality of circular templates on the surface of the substrate to generate the plurality of concatemers immobilized to the substrate. The amplification may comprise RCA. The plurality of circular templates may comprise different template inserts. The method may further comprise circularizing a plurality of linear templates comprising different template inserts using splint molecules and ligating the respective two ends of the plurality of linear templates to generate the circular templates.

The method may further comprise loading a plurality of linear templates on the substrate, wherein the substrate comprises a plurality of surface primers, to hybridize the plurality of linear templates to the plurality of surface primers, ligating the respective two ends of the plurality of linear templates to generate a plurality of circular templates, and using the plurality of surface primers to amplify the plurality of circular templates to generate the plurality of concatemers immobilized to the substrate. The amplification may comprise RCA. The plurality of linear templates may comprise different template inserts.

A concatemer may comprise any useful number of copies of a template insert or repeating units. For example, a concatemer may comprise about, at least about, and/or at most about 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3500, 4000, 4500, 5000, 5500, 6000, 6500, 7000, 7500, 8000, 8500, 9000, 9500, 10,000, or more copies.

The density of concatemers on the substrate, once immobilized, may be any useful density. In some cases, the density may be optimized to be higher than a predetermined threshold to ensure high throughput. In some cases, the density may be optimized to be lower than a second predetermined threshold to ensure that sequencing signals of individual concatemers are resolvable and/or neighboring concatemers do not hinder sequencing reactions. In some cases, the surface chemistry may be tailored to obtain a desired concatemer density. For example, the density may be about, at least about, and/or at most about an order of 10², 10³, 10⁴, 10⁵, 10⁶, 10⁷, 10⁸ concatemers per square millimeter (concatemers/mm²). The predetermined threshold and/or second predetermined threshold may be any of the above orders of magnitudes. In some cases, the density may be about, at least about, and/or at most 1.0×10⁵, 1.5×10⁵, 2.0×10⁵, 2.5×10⁵, 3.0×10⁵, 3.5×10⁵, 4.0×10⁵, 4.5×10⁵, 5.0×10⁵, 5.5×10⁵, 6.0×10⁵, 6.5×10⁵, 7.0×10⁵, 7.5×10⁵, 8.0×10⁵, 8.5×10⁵, 9.0×10⁵, 9.5×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷ concatemers/mm². The predetermined threshold and/or second predetermined threshold may be any of the above numeric values. The pitch of the concatemers (e.g., center-to-center distance of neighboring concatemers) on the substrate, once immobilized, may be any useful pitch. For example, the pitch may be about, at least about, and/or at most about 0.1, 0.15, 0.2, 0.25, 0.3, 0.35, 0.4, 0.45, 0.5, 0.55, 0.6, 0.7, 0.75, 0.8, 0.85, 0.9, 0.95, 1.0, 1.1, 1.2, 1.3, 1.4, 1.5, 1.6, 1.7, 1.8, 1.9, 2.0 or more micrometers. In some cases, a substrate may comprise any number of concatemers, such as comparable to the number of individually addressable locations described elsewhere herein. For example, a substrate may immobilize at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more concatemers.

### Computer Systems

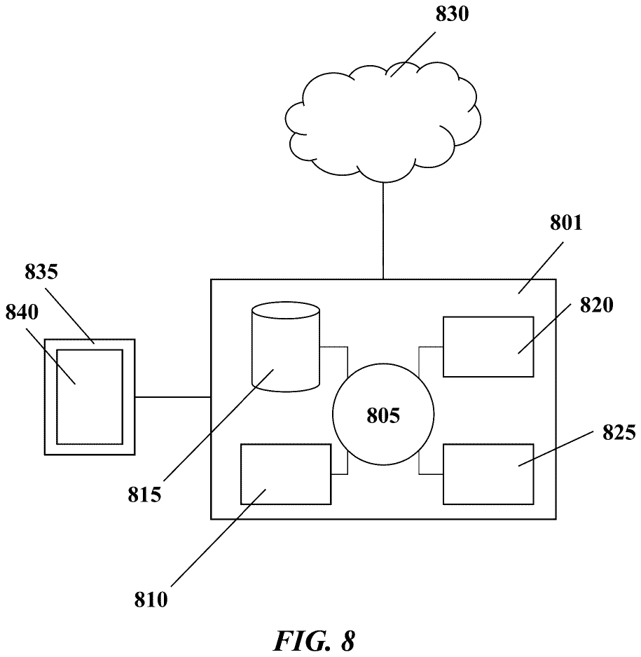

The present disclosure provides computer control systems that are programmed to implement methods of the disclosure. FIG. 8 shows a computer system 801 that is programmed or otherwise configured to implement methods of the disclosure, such as to control the systems described herein (e.g., reagent dispensing, detecting, etc.) and collect, receive, and/or analyze sequencing information. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an isolated or substantially isolated internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries, and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, provide examples: e.g., results of a nucleic acid sequence (e.g., sequence reads). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, spatially resolve a plurality of analyte sequences using sequencing information.

### EXAMPLES

These examples are provided for illustrative purposes only and are not intended to limit the scope of the claims provided herein.

### Example 1: Preparing Substrate with PEG Spacers

A first type of substrate surface is prepared by depositing azidosilane on a wafer via chemical vapor deposition (CVD) to yield an azide surface. In a first layer, DBCO-PEG10-DBCO molecules are attached via azide-DBCO click chemistry, at 100 μM at 45° C. for 30 minutes to 2 hours. In a second layer, 3-arm-azide-PEG5 molecules are attached via azide-DBCO click chemistry, at 100 μM at 45° C. for 30 minutes to 2 hours. Next, surface primers are conjugated to the surface via click chemistry of DBCO-primers, at 100 μM at 45° C. for 30 minutes to 2 hours.

A second type of substrate surface is prepared by depositing azidosilane on a wafer via chemical vapor deposition (CVD) to yield an azide surface. In a first layer, DBCO-PEG2 k-DBCO molecules are attached via azide-DBCO click chemistry, at 100 μM at 45° C. for 2 hours. In a second layer, azide-PEG2 k-azide molecules are attached via azide-DBCO click chemistry, at 100 μM at 45° C. for 2 hours. Next, surface primers are conjugated to the surface via click chemistry of DBCO-primers, at 100 μM at 45° C. for 2 hours.

A third type of substrate surface is prepared by depositing azidosilane on a wafer via chemical vapor deposition (CVD) to yield an azide surface. In a first layer, DBCO-PEG5 k-DBCO molecules are attached via azide-DBCO click chemistry, at 100 μM at 45° C. for 30 minutes to 2 hours. Next, surface primers are conjugated to the surface via click chemistry of azide-primers, at 100 μM at 45° C. for 30 minutes to 2 hours.

### Example 2: Addition of Ethylene Carbonate During RCA

The effect of adding ethylene carbonate (EC) during rolling circle amplification was investigated by providing each of 0% EC (control), 2% EC, 5% EC, and 8% EC during RCA in solution, depositing the RCA product concatemers (e.g., nanoballs) to a substrate coupon (e.g., a section of a substrate), and probing the RCA concatemers with labeled sequencing primer comprising an Atto532 label. An E. Coli library was used.

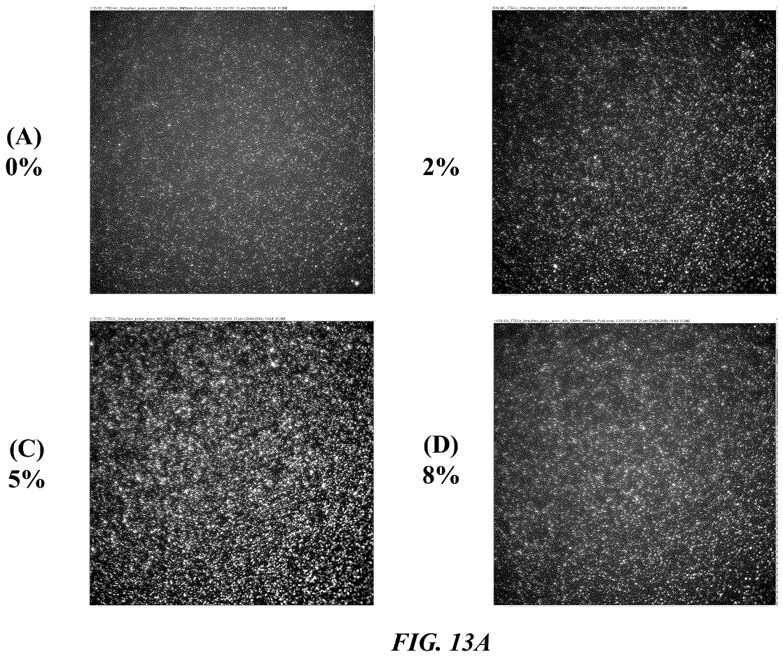

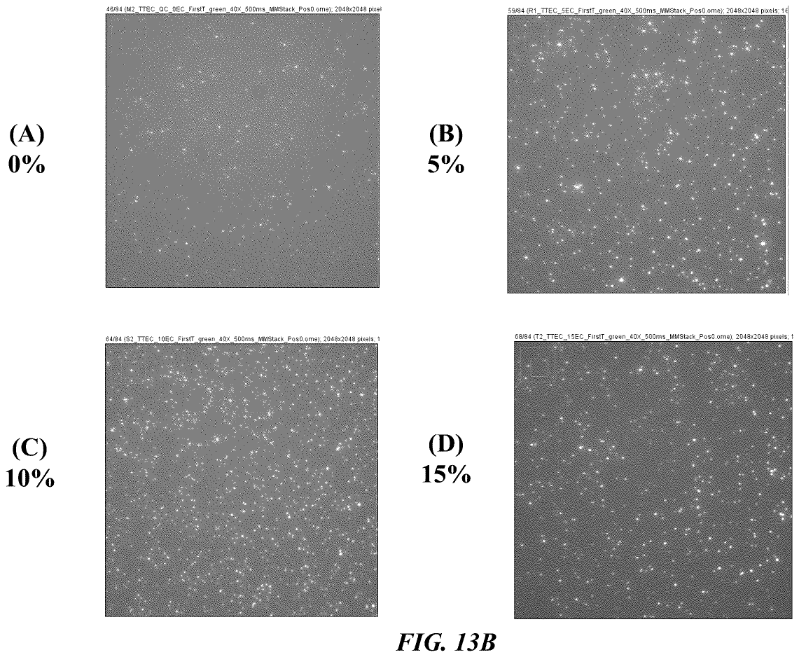

A 2048×2048 pixel image of the Atto532 probe signals for each assay is depicted in FIG. 13A, panels (A), (B), (C), and (D) for 0% EC (control), 2% EC, 5% EC, and 8% EC respectively. It can be seen that the signals are significantly improved and distinct in the Panel (C) image (5% EC), particularly over the Panel (A) image (for the control). The mean signals from the probing provided in Table 1 below also show that the addition of 5% EC during in solution RCA resulted in a gain in approximately 230% more signal compared to the control (no EC addition).

TABLE 1

| % of Ethylene | Mean Signal/Colony (A.U.) Atto532 |
|---|---|
| Carbonate | Probing |
| 0% | 275 |
| 2% | 478 |
| 5% | 639 |
| 8% | 408 |

### Example 3: Addition of Ethylene Carbonate for Sequencing Primer Hybridization

The effect of adding ethylene carbonate (EC) in sequencing primer hybridization was investigated by providing each of 0% EC (control), 5% EC, 10% EC, and 15% EC during sequencing primer hybridization with RCA product concatemers (e.g., nanoballs) on a substrate coupon, and detecting the signal after a first labeled T base dNTP is incorporated. An E. Coli library was used.

A 2048×2048 pixel image of the probe signals for each assay is depicted in FIG. 13B, panels (A), (B), (C), and (D) for 0% EC (control), 5% EC, 10% EC, and 15% EC respectively. It can be seen that the signals are significantly improved in each of the Panels (B), (C), and (D) images (5% EC, 10% EC, and 15% EC respectively), over the Panel (A) image (for the control). The mean signals from the probing provided in Table 2 below also show that the addition of EC during sequencing primer hybridization resulted in a significant gain in signal compared to the control (no EC addition).

TABLE 2

| % of Ethylene | Mean Signal/Colony (A.U.) First T |
|---|---|
| Carbonate | Incorporation |
| 0% | 694 |
| 5% | 1013 |
| 10% | 2659 |
| 15% | 2634 |

### Example 4: On-Surface RCA Sequencing

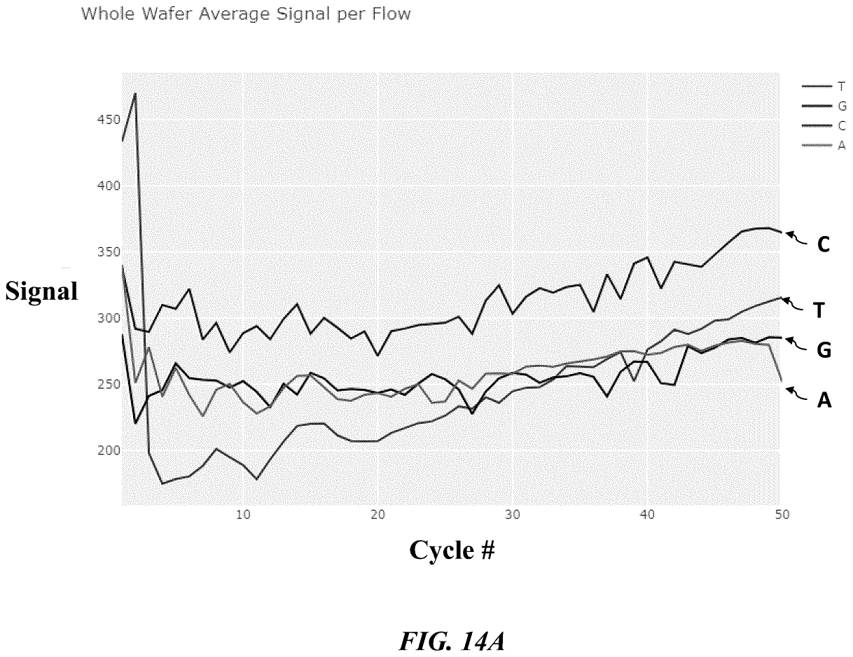

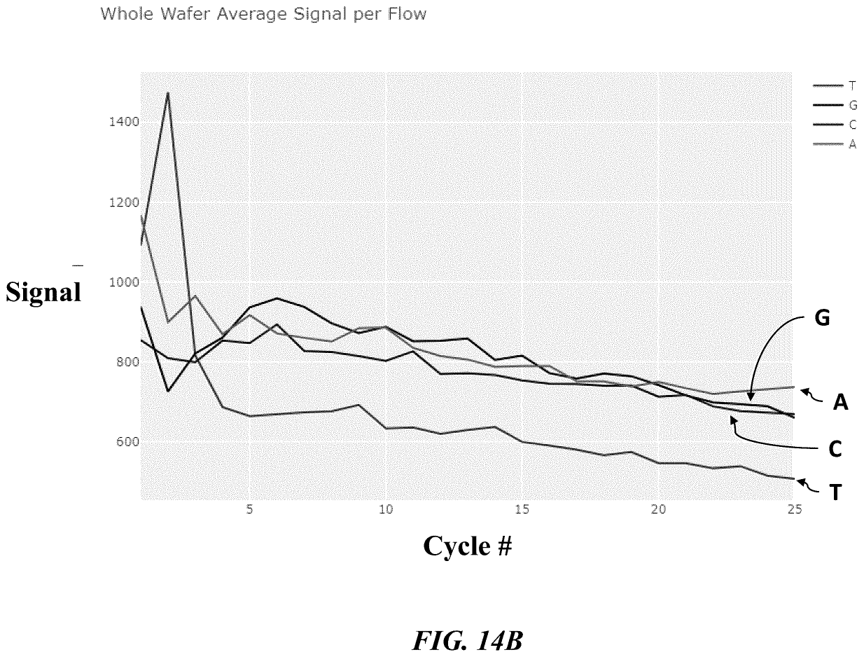

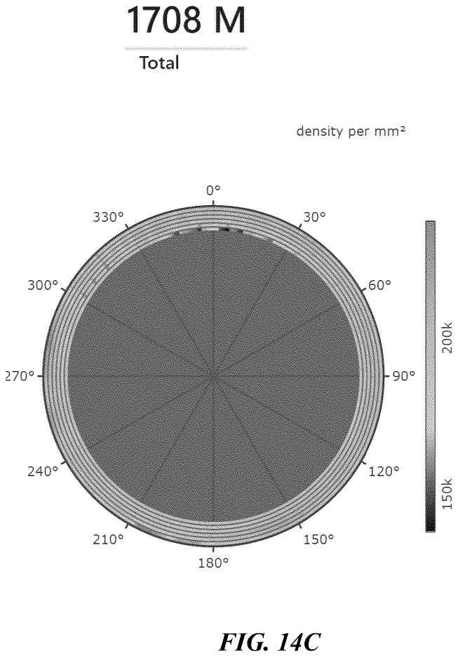

The general workflow according to FIG. 10M was performed, comprising depositing of circular templates onto the surface, performing on-surface RCA, and sequencing the RCA concatemer products. The sequencing results are presented in FIG. 14A, showing the average signal per flow for each base (T, G, C, A). Table 3 shows the phasing metrics.

TABLE 3

| Base | Lag, Effective | Lead, Effective |
|---|---|---|
| T | 0.211 | 0.597 |
| G | 0.117 | 0.438 |
| C | 0.108 | 0.461 |
| A | 0.238 | 0.449 |
| Average | 0.132 | 0.469 |

The template inserts were successfully detected in the sequencing results with effective lead and lag metrics below 0.5 (with 1 being maximum & worst quality, 0 being minimum & best quality).

### Example 5: Directly Functionalizing Nitride Surfaces for Bead Capture

A simple and efficient method of surface functionalization may begin with a nitride surface (e.g., thereby reducing the number of steps required to produce a surface capable of capturing biological analytes). Beneficially, multiple types of bonds can be formed on surfaces with amine reactive sites. Nitride surfaces can be generally functionalized by cleaning with isopropyl alcohol and oxygen plasma and then exposing to hydrogen fluoride. Hydrogen fluoride may convert Si₃N₄ on the surface into Si—NHₓ bonds. In some cases, nitrides can form a native oxide on their surface which can be functionalized by a silane reaction. This oxide layer can be removed from some or all of the nitride surface in order to work directly with the surface amines. Amino groups on the surface can be used to immobilize biomolecules or be functionalized directly by amine-based surface reactions including but not limited to NHS-ester chemistry or conversion to an azide surface. In some cases, a nitride surface may comprise at least one of SiN/Si₃N₄, TiN, GaN, Ge₃N₄.

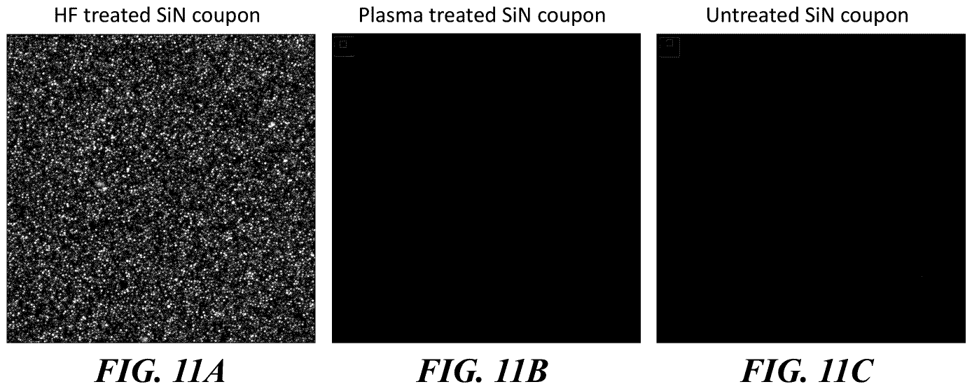

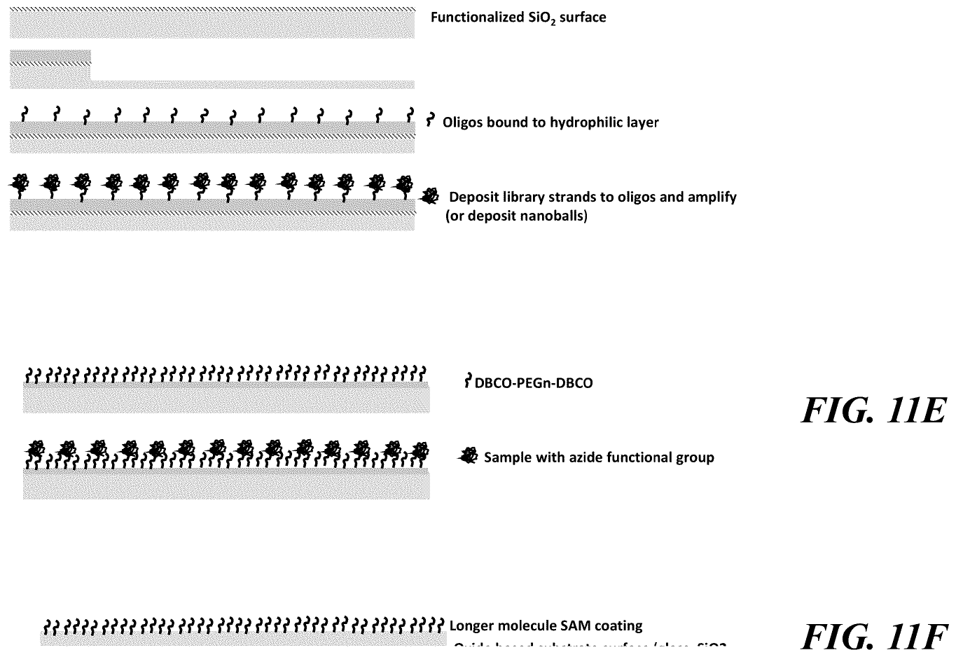

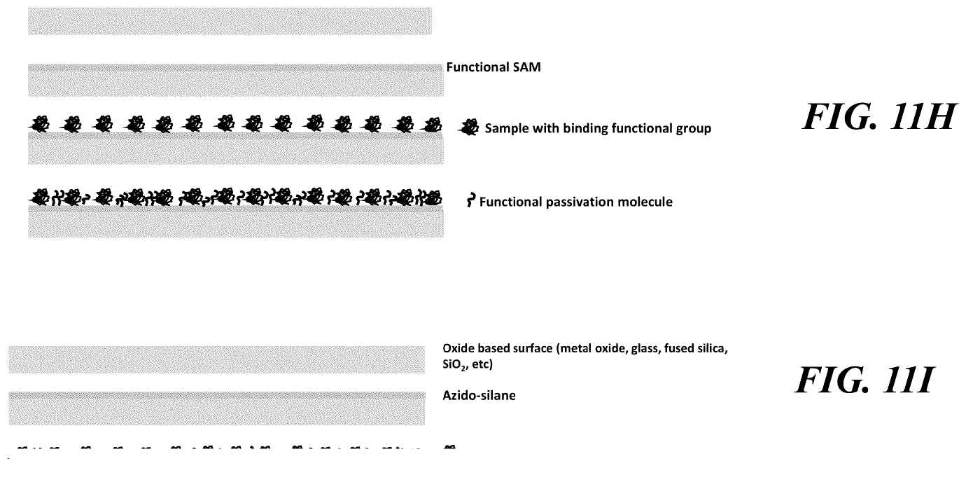

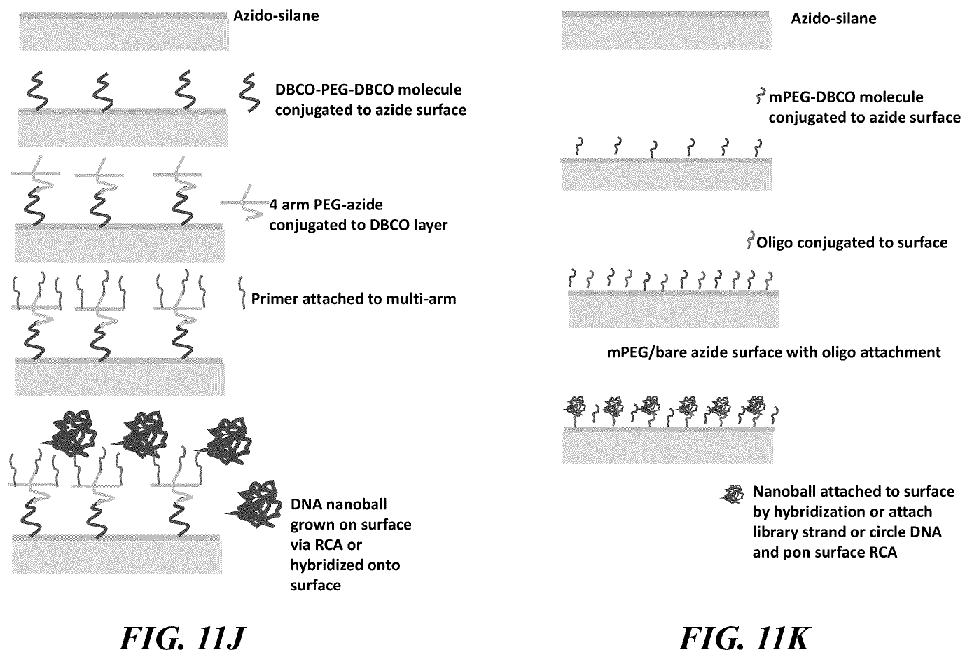

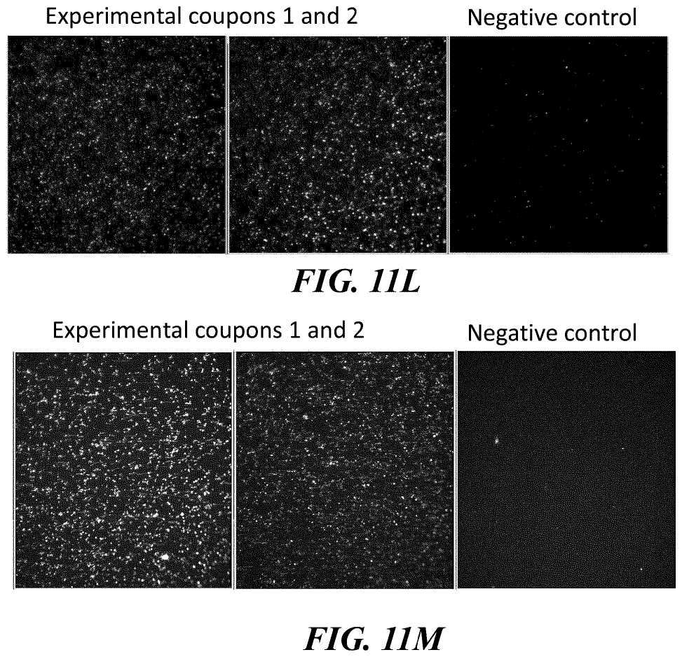

By way of example, as shown in FIGS. 11A-11C, nitride-functionalized surfaces are prepared and tested for capacity to capture sequencing beads. Unpatterned silicon nitride substrate (e.g., coupons) was prepared. Coupons were cleaned with isopropyl alcohol and exposed to oxygen plasma. The cleaned coupon in FIG. 11A was immersed in dilute hydrogen fluoride (i.e., to convert Si₃N₄ into Si—NHₓ), then rinsed and dried. The cleaned coupon in FIG. 11B was merely rinsed and dried without hydrogen fluoride treatment. The coupon in FIG. 11C was completely untreated (i.e., not cleaned and not functionalized). After initial preparation, the coupons were exposed to beads comprising fluorescent labels. Sequencing beads (e.g., as described elsewhere herein) were only captured by the coupon treated with hydrogen fluoride, as shown by the fluorescence in FIG. 11A (e.g., fluorescently labeled oligos hybridized to sequencing beads coupled to the substrate). The coupons in FIGS. 11A-11C are unpatterned.

A primary chemistry for selectively binding to the surface amines on the exposed nitride surfaces is NHS-ester and ester-based conjugation chemistries for binding to amines. The positively charged nature of an amine surface may also be used to bind negatively charged molecules or particles such as amplified DNA beads or RCA-DNA-clusters (e.g., nanoballs) directly rather than indirectly via sequencing beads. Beneficially, surface amines can be converted to surface azide groups, which can then participate directly in click chemistry and copper free click chemistry reactions.

### Example 6: Priming Substrates for Analyte Capture

### Generic Hydrophilic Layer

FIG. 11D illustrates a simple schema for providing a hydrophilic layer that can be used for biological analyte attachment (e.g., for attachment of nanoballs or library molecules prior to amplification).

An unpatterned oxide-based surface (e.g., SiO₂) may be silane-functionalized (e.g., SiH₄ bonds) via a self-assembly monolayer (SAM) process. Examples of SAM functionalization can be found for example in “Fabrication of patterned silane based self-assembly monolayers by photolithography and surface reactions on silicon-oxide substrates” (2010) Herzer, Hoeppener, and Schubert. Chem. Commun., 46, 5634-5652, which is hereby incorporated by reference in its entirety.

A hydrophilic material may be overlaid upon the silane-functionalized surface. In some cases, a hydrophilic material may comprise acrylic polymers (e.g., acrylic, acrylonitrile, acrylamide, and maleic anhydride polymers), ether polymers (e.g., polyesters, polyurethanes, and poly(propylene glycol)), fluorocarbon polymers, polystyrene polymers, poly(vinyl chloride) (PVC) polymers, poly(n-vinylpyrrolidone) (PVP) polymers, or a combination thereof. More specifically, a hydrophilic material may comprise polyethylene glycol (PEG), polyethylenimine (branched or unbranched), poly(acrylic acid), poly(sodium 4-styrenesulfonate), poly(allylamine hydrochloride), sodium polyacrylate (cross-linked or not), poly(vinyl sulfonic acid), poly(vinyl sulfate), poly(2-ethyl-2-oxazoline), polyacrylamide, poly(allylamine), poly(vinyl alcohol-co-ethylene), poly(4-styrenesulfonic acid-co-maleic acid), poly(acrylamide-co-acrylic acid), Poly(2-(dimethylamino)ethyl methacrylate), Poly(acrylamide-co-diallyldimethylammonium chloride), Poly(styrene-alt-maleic acid), Poly(2-dimethylamino)ethyl methacrylate) methyl chloride, Poly(methyl vinyl ether), Poly(methyl vinyl ether-alt-maleic acid), Poly(N-isopropylacrylamide), Poly(methacrylic acid), Poly(N-isopropylacrylamide-co-methacrylic acid), Poly(2-(diisopropylamino)ethyl methacrylate), polyvinyl alcohol (PVA), Poly(N-isopropylacrylamide-co-butyl acrylate), poly(2-propyl-2-oxazoline), poly(2-methyl-2-oxazoline), Poly(N-(2-hydroxypropyl) methacrylamide), Poly(N,N-dimethylacrylamide), Poly(N-isopropylacrylamide-co-acrylamide), Poly(N-isopropylacrylamide), N-hydroxysuccinimide (NHS) ester, Poly(2-propylacrylic acid), Cucurbit[5]uril hydrate, Cucurbit[7]uril hydrate, Cucurbit[8]uril hydrate, Poly(methyl vinyl ether-alt-maleic anhydride), Poly(methyl vinyl ether-alt-maleic acid monoethyl ester), Polyanetholesulfonic acid, Poly[bis(2-chloroethyl) ether-alt-1,3-bis[3-(dimethylamino)propyl]urea] quaternized, Poly(4-styrenesulfonic acid), or a combination thereof. It will be appreciated that other hydrophilic polymers known in the art may be used alternatively or in addition to those listed above. Alternatively or in addition, other hydrophilic materials may be used (e.g., polar or charged molecules).

In some cases, the hydrophilic polymer further comprises oligo attachment sites (e.g., DBCO or NHS-ester or primers). Oligos (e.g., comprising a region of sequence complementarity to nucleotide analytes) may be coupled to oligo attachment sites (e.g., via click-chemistry or hybridization). Library molecules may be provided, where surface-coupled oligos may capture library molecules for on surface amplification). Alternatively, nanoballs (e.g., already amplified library molecules) may be provided, where surface-coupled oligos may capture nanoballs.

In some cases, one or more of hydrophilic polymer deposition or oligo coupling may be repeated one or more times to provide multiple layers of said material on the surface of a substrate or to ensure coverage of the surface at a desired density. In some cases, library molecules or nanoballs may be provided one or more times.

### PEG-Based Layer

In some cases, one of either an oligo attachment layer or a hydrophilic layer may be unnecessary. For example, as illustrated in FIGS. 11E-11G, analytes may be directly attached to functional groups on the surface (e.g., not via hybridization to oligo(s)).

FIGS. 11E and 11F illustrate direct attachment of PEG-DBCO moieties to a surface. In each case, an oxide-based substrate is coated via SAM. In FIG. 11E, the SAM coating comprises azido-silane. PEGₙ molecules (where the PEG may be of any useful length and/or molecular weight) that are functionalized with DBCO at either end are provided, where the PEGₙ molecules may be click coupled to the azido-silane. Biological analytes (e.g., library molecules or nanoballs) with azido functional groups may be coupled to the PEG layer via click chemistry. In FIG. 11F, the SAM coating comprises azido-PEGₙ-silane (e.g., where the PEG may be of any useful length and/or molecular weight). Biological analytes (e.g., library molecules or nanoballs) with DBCO functional groups may be coupled to the PEG layer via click chemistry.

### Direct Attachment to Substrate

FIG. 11G illustrates direct attachment of analyte molecules to a surface (e.g., without intervening hydrophilic or PEG layers). In this case, the SAM layer itself comprises capture moieties (e.g., the SAM layer is azido functionalized). Biological analytes that are DBCO-functionalized may be conjugated to the surface via click chemistry.

Any combination of azido/DBCO functional groups may be used in the above schemes. It will also be appreciated that other suitable types of chemical attachments may be used in place of click chemistry.

### Example 7: Surface Passivation

To enhance signal-to-noise ratio (SNR) for downstream processes (e.g., to improve detection of nucleotide incorporation in sequencing reactions), it may be beneficial to a) control the number of attachment sites on a substrate (e.g., to provide sufficient distance on average between biological analytes for high image resolution), or b) after loading, prevent additional analyte attachment to a surface. These general methods may be termed ‘surface passivation.’ In some cases, this may be performed on any of the substrates described herein.

### Post-Loading Passivation

Schemes for passivating a surface after biological analytes have been loaded onto the surface are illustrated in FIG. 11H. As with the other surface preparation schemes described herein, a substrate (e.g., an oxide-based substrate surface such as glass, SiO₂, metal oxide, etc.) may be SAM coated. The SAM coating may be deposited by either vapor or liquid phase. The SAM coating may be functionalized with a conjugation moiety (e.g., thiol, carboxy, maleimide, strained alkyne, azide, amine, ester, NHS, etc.). As described elsewhere herein, analytes may be loaded onto the functionalized surface and captured by the functional moieties (e.g., conjugated to the surface via click chemistry).

In some cases, a SAM coating may be deposited with an electrostatic charge (e.g., where the SAM molecules comprise a polar or charged moiety (e.g., an amine)). The charge may be opposite of that of analytes to be loaded. For example, the SAM electrostatic charge may be positive if the analyte molecules are negatively charged DNA. Azide moieties on a substrate surface may be converted into amine groups (e.g., by exposure to a reducing agent (e.g., DTT or TCEP)). Amine groups are positively charged which can help to stabilize loaded nanoballs (or other negatively charged analytes). In some cases, modifying surface charge may also be beneficial for spacing loaded analytes on the surface (e.g., by providing some density of charge repulsion moieties on the substrate and thus reducing the density of analytes during conjugation).

After loading analytes, a passivating molecule (e.g., PEG) may be conjugated to the surface. By way of example, the substrate may be amine-functionalized, analytes may be DBCO-functionalized, the PEG molecules may be DBCO-functionalized, and the analytes and PEG may be attached to the surface via click chemistry.

### Concurrent Loading and Passivation

FIG. 11I illustrates an example where analytes and passivation molecules may be loaded onto a substrate simultaneously. By way of example, a substrate (e.g., an oxide-based substrate surface such as glass, SiO₂, metal oxide, etc.) may be SAM coated (e.g., with a silane-functionalized SAM coating). Analytes and passivation molecules (e.g., PEG) will comprise the same type of functional moieties (e.g., DBCO). Analytes and passivation molecules are loaded together onto the substrate. By modulating the ratio of analytes to passivation molecules, it is possible to control the average density of analytes (e.g., the average center-to-center distance between adjacent analytes). In concurrent loading, passivation molecules may serve as a crowding agent. In some cases, passivation molecules may buffer analytes from other analytes (e.g., to prevent or reduce analyte clumping in solution or after loading).

### Pre-Loading Passivation

Alternatively, or in addition, surfaces may be passivated prior to loading (e.g., the surface may be coated with a non-hydrophilic material). The passivated surface may comprise analyte capture moieties. Controlling the concentration of passivation molecules that are loaded onto a surface, affects the density of passivation molecule coupled to the surface. This influences the amount of coupling moieties available for analyte binding. A pre-loading passivated surface may be further subjected to post-loading passivation after the addition of analytes, as described above. Alternatively or in addition, a pre-loading passivated surface may be concurrently loaded with analyte and further passivation molecules. In some cases, a pre-loading passivated surface that is concurrently loaded with passivation molecules and analytes may be further subjected to post-loading passivation.

In any of the passivation methods, one or more of the passivation loading and analyte loading steps may be repeated multiple times in any combination. In one example, a surface may be loaded with analytes at a first density, partially passivated, loaded with analytes to a second density (i.e., to increase the total density of analytes on the surface), and passivated again. Such a series of loadings may serve to reduce analyte clumping. In some cases, multiple types of passivating molecules may be used, for example, multiple different molecular weight PEGs or a combination of linear and branched PEG molecules. In some cases, PEG molecules used for passivation may be of a similar molecular weight or size as the analytes (e.g., PEG of 2000 g/mol may be used to passivate a surface loaded with oligonucleotide primers).

It will be understood that the loading methods, passivation methods, and surface functionalization methods described here may be used in any combination.

### Example 8: Nanoball Loading with PEG-Functionalized Surfaces

### Loading Schema

As described above, in some cases, a surface may comprise multiple functionalization layers, passivation layers, functionalization moieties, passivation molecules, or combinations thereof. FIGS. 11J and 11K illustrate specific examples of surfaces with PEG molecules used for nanoball loading.

In FIG. 11J, an azido-silane functionalized substrate is conjugated to DBCO-PEG-DBCO molecules via click chemistry. An additional layer of branched PEG (e.g., 4-arm PEG) that is azide functionalized is conjugated to the PEG-DBCO moieties via click chemistry The molecular weight of the PEG in the unbranched or branched layers can be varied. DBCO-functionalized oligos are provided to the substrate for conjugation via click chemistry. A majority of the oligos will be conjugated to branched PEG molecules. One or more oligos may be conjugated to azide moieties on the surface of the substrate. The prepared substrate may be loaded with DNA nanoballs. Alternatively, the prepared substrate may be loaded with library molecules which can then be amplified on the surface.

Azide-functionalized oligos may, alternatively, be conjugated to PEG-DBCO moieties (e.g., in the absence of the branched PEG molecules).

FIG. 11K illustrates a combination of PEG passivation and oligo loading on a substrate surface. As elsewhere herein, an azido-silane functionalized substrate may be conjugated to DBCO-PEG molecules and to DBCO-oligonucleotides. In some cases, PEG molecules may be loaded first and then oligos loaded subsequently. In some cases, oligos may be loaded first and then PEG molecules loaded subsequently. In some cases, PEG molecules and oligos may be loaded simultaneously or substantially simultaneously. In FIG. 11K, the PEG molecules are methyl-PEG (m-PEG). m-PEGs are hydrophobic and may serve as surface passivation. Alternatively, or in addition, other passivation molecules may be used, as described elsewhere herein (e.g., other non-hydrophilic molecules).

In some cases, one or more reference beads may be conjugated concurrent with or subsequent to oligo conjugation.

Click chemistry is ideal for substrate preparation as it can be performed in aqueous solutions, which is an amenable environment for biological analytes. In addition, click chemistry is very rapid and is not susceptible to cross-reactions. In all click chemistry reactions described herein, azide groups may be substituted with DBCO moieties (e.g., a surface may be DBCO-functionalized and oligos may be azido-functionalized). However, other attachment chemistries (e.g., NHS ester) may be used in place of or in addition to click chemistry.

### Experimental Results

Two conditions were tested for nanoball loading. In condition 1 (FIG. 11L and “no PEG” in Table 4), a substrate was loaded with oligos (where the oligos had sequence complementarity to a region in DNA analytes). In condition 2 (FIG. 11M and “primer/PEG” in Table 4), a substrate was loaded with a 50:50 ratio of PEG2K: oligos, where PEG2K indicates PEG molecules with an average molecular weight of 2,000 g/mol. In each condition, circular DNA templates were incubated with the prepared substrates for 30 minutes at 45° C. During an additional 30 minutes at 45° C., RCA was performed, resulting in the production of tethered nanoballs on each surface. In each of FIGS. 11L and 11M, the first two panels correspond to coupons where conditions 1 and 2 were performed, respectively. The third panel in each figure corresponds to a negative control (e.g., no circular DNA present during incubation/amplification). As shown in Table 4, both conditions result in the successful production of nanoballs (e.g., counted particles). Each row corresponds to a location on one of the panels in FIG. 11L or 11M, respectively. After RCA, each coupon was loaded with sequencing primers that hybridized to nanoballs, and a single primer extension step was performed (e.g., where labeled U nucleotide is incorporated into hybridized primers). In Table 4, median RFU (relative fluorescence units) refers to measurements of these labels and indicates the presence of nanoballs on each surface. As can be seen from the figures and table, the presence of PEG passivation molecules did not hinder the attachment of DNA template, amplification on surface, or incorporation reactions into DNA nanoballs.

TABLE 4
Nanoball formation from on-surface RCA,
with or without PEG passivation molecules

| Condition | particle count | median RFU |
|---|---|---|
| Primer | 19460 | 604 |
| Primer | 37193 | 2978 |
| Primer | 16770 | 253 |
| Primer | 8210 | 348 |
| Primer | 5704 | 250 |
| Primer | 9933 | 501 |
| Primer | 10527 | 190 |
| Primer | 8591 | 276 |
| Primer | 8005 | 414 |
| PEG/primer | 4666 | 231 |
| PEG/primer | 5966 | 213 |
| PEG/primer | 5775 | 188 |
| PEG/primer | 6521 | 190 |
| PEG/primer | 6269 | 151 |
| PEG/primer | 6897 | 376 |
| PEG/primer | 5872 | 346 |
| PEG/primer | 7936 | 188 |
| PEG/primer | 8153 | 218 |

### Example 9: Sequencing RCA Products

The general workflow according to FIG. 10L was performed, comprising performing RCA in solution to generate concatemers, depositing the concatemers onto the surface, and sequencing the RCA concatemer products. In this assay, the sequencing primers were hybridized to the concatemers prior to loading the concatemers onto the substrate. The sequencing results are presented in FIG. 14B, showing the average signal per flow for each base (T, G, C, A). FIG. 14C shows a density map of the loaded concatemers on the substrate, with the gradation indicating count/mm² on the wafer. A total of 1.7 billion concatemers were identified with relatively even loading (approximately 180,000˜200,000 concatemers/mm² density) across the entire substrate. The template inserts, comprising E. Coli library, were successfully detected in the sequencing results.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

Various embodiments are described in the following clauses.

Clause 1. A method for sequencing concatemers, comprising: providing a substrate comprising a plurality of concatemers immobilized thereto; extending sequencing primers hybridized to the plurality of concatemers in a plurality of extension steps; and detecting signals, or lack thereof, from the substrate during or subsequent to at least a subset of the plurality of extension steps to determine sequencing reads corresponding to the plurality of concatemers, wherein the substrate is rotated about a rotational axis (i) during or subsequent to one or more extension step(s) of the plurality of extension steps, (ii) during the detecting, or both (i) and (ii).

Clause 2. The method of clause 1, wherein the extending of the sequencing primers comprises providing, in the extension step, a plurality of nucleotides under conditions sufficient to incorporate the plurality of nucleotides, wherein at least a subset of the plurality of nucleotides is labeled, and wherein in (c) the signals, or lack thereof, are indicative of incorporation of labeled nucleotides of the plurality of nucleotides.

Clause 3. The method of clause 2, wherein all of the plurality of nucleotides are labeled.

Clause 4. The method of clause 2, wherein at most 50% of the plurality of nucleotides are labeled.

Clause 5. The method of clause 4, wherein at most 10% of the plurality of nucleotides are labeled.

Clause 6. The method of any one of clauses 2-5, wherein the plurality of nucleotides comprise a single canonical base type selected from A, T, U, G, or C.

Clause 7. The method of any one of clauses 2-5, wherein the plurality of nucleotides comprises two or three canonical base types.

Clause 8. The method of any one of clauses 2-5, wherein the plurality of nucleotides comprises four canonical base types.

Clause 9. The method of any one of clauses 8-9, wherein the labeled nucleotides of the plurality of nucleotides are detectable at a same wavelength or wavelength range.

Clause 10. The method of any one of clauses 8-9, wherein the labeled nucleotides of the plurality of nucleotides are detectable at different wavelengths or wavelength ranges.

Clause 11. The method of any one of clauses 2-10, wherein the plurality of nucleotides comprises reversibly terminated nucleotides.

Clause 12. The method of any one of clauses 2-10, wherein the plurality of nucleotides comprises non-terminated nucleotides.

Clause 13. The method of any one of clauses 1-12, wherein the substrate is patterned.

Clause 14. The method of any one of clauses 1-12, wherein the substrate is unpatterned.

Clause 15. The method of any one of clauses 1-14, wherein the substrate is aminated.

Clause 16. The method of any one of clauses 1-14, wherein the substrate comprises azido groups.

Clause 17. The method of any one of clauses 1-16, wherein the substrate comprises a polyethylene glycol (PEG) spacer.

Clause 18. The method of clause 17, wherein the substrate comprises a layer of the PEG spacer.

Clause 19. The method of any one of clauses 17-18, wherein the PEG spacer comprises a multi-arm PEG molecule.

Clause 20. The method of any one of clauses 17-19, wherein the PEG spacer comprises a methyl terminated PEG molecule.

Clause 21. The method of any one of clauses 1-20, wherein the substrate comprises a plurality of surface primers that are covalently bound to the substrate.

Clause 22. The method of clause 21, wherein a plurality of concatemers are hybridized to the plurality of surface primers, wherein the plurality of surface primers comprises the sequencing primers.

Clause 23. The method of any one of clauses 1-22, wherein the substrate is rotated about a rotational axis during or subsequent to one or more extension step(s) of the plurality of extension steps.

Clause 24. The method of any one of clauses 1-23, wherein the substrate is rotated about a rotational axis during the detecting.

Clause 25. The method of clause 24, wherein the substrate is rotated about a rotational axis (i) during or subsequent to one or more extension step(s) of the plurality of extension steps, and (ii) during the detecting.

Clause 26. The method of any one of clauses 1-25, further comprising, prior to (a), loading the plurality of concatemers on the substrate to immobilize the plurality of concatemers on the substrate.

Clause 27. The method of clause 26, wherein the substrate comprises a plurality of surface primers that are hybridized to the plurality of concatemers, wherein the plurality of surface primers comprises the sequencing primers.

Clause 28. The method of clause 26, wherein the substrate is absent of surface primers.

Clause 29. The method of clause 28, further comprising contacting the sequencing primers to the plurality of concatemers subsequent to immobilizing the plurality of concatemers on the substrate.

Clause 30. The method of clause 28, further comprising contacting the sequencing primers to the plurality of concatemers prior to loading the plurality of concatemers on the substrate.

Clause 31. The method of any one of clauses 26-30, further comprising, prior to loading the plurality of concatemers on the substrate, amplifying a plurality of circular templates in solution to generate the plurality of concatemers, wherein the plurality of circular templates comprises different nucleic acid template inserts.

Clause 32. The method of clause 31, wherein the amplifying comprises rolling circle amplification (RCA).

Clause 33. The method of any one of clauses 31-32, further comprising, prior to amplifying the plurality of circular templates, circularizing a plurality of linear templates comprising the different nucleic acid template inserts using splint molecules and ligating the respective two ends of the plurality of linear templates to generate the circular templates.

Clause 34. The method of clause 33, wherein the splint molecules are used to amplify the plurality of circular templates.

Clause 35. The method of any one of clauses 1-25, further comprising, prior to (a), loading a plurality of circular templates on the substrate, wherein the substrate comprises a plurality of surface primers; hybridizing the plurality of circular templates to the plurality of surface primers; and using the plurality of surface primers to amplify the plurality of circular templates on the substrate to generate the plurality of concatemers immobilized to the substrate, wherein the plurality of circular templates comprises different nucleic acid template inserts.

Clause 36. The method of any one of clauses 1-25, further comprising, prior to (a), loading a plurality of circular templates on the substrate, wherein the plurality of circular templates are hybridized to a plurality of primers that are conjugated to reactive moieties, and wherein the substrate comprises additional reactive moieties configured to couple to the reactive moieties, to bind the plurality of circular templates to the substrate, and using the plurality of primers to amplify the plurality of circular templates on the substrate to generate the plurality of concatemers immobilized to the substrate, wherein the plurality of circular templates comprises different nucleic acid template inserts.

Clause 37. The method of any one of clauses 35-36, wherein the amplifying comprises rolling circle amplification (RCA).

Clause 38. The method of any one of clauses 35-37, further comprising circularizing a plurality of linear templates comprising the different nucleic acid template inserts using splint molecules and ligating the respective two ends of the plurality of linear templates to generate the plurality of circular templates.

Clause 39. The method of any one of clauses 1-25, further comprising, prior to (a), loading a plurality of linear templates on the substrate, wherein the substrate comprises a plurality of surface primers, to hybridize the plurality of linear templates to the plurality of surface primers, ligating the respective two ends of the plurality of linear templates to generate a plurality of circular templates, and using the plurality of surface primers to amplify the plurality of circular templates to generate the plurality of concatemers immobilized to the substrate, wherein the plurality of linear templates comprises different nucleic acid template inserts.

Clause 40. The method of clause 39, wherein the amplifying comprises rolling circle amplification (RCA).

Clause 41. The method of any one of clauses 1-40, further comprising (d) extending the sequencing primers hybridized to the plurality of concatemers to generate a plurality of second concatemers, (e) hybridizing second sequencing primers to the plurality of second concatemers, (f) extending the second sequencing primers in a second plurality of extension steps, and (g) detecting second signals, or lack thereof, from the substrate during or subsequent to at least a subset of the second plurality of extension steps to determine second sequencing reads corresponding to the plurality of concatemers.

Clause 42. The method of clause 41, further comprising processing the sequencing reads and the second sequencing reads as paired end reads.

Clause 43. The method of any one of clauses 1-40, further comprising (d) amplifying the plurality of concatemers to generate a plurality of second concatemers, (e) hybridizing second sequencing primers to the plurality of second concatemers, (f) extending the second sequencing primers in a second plurality of extension steps, and (g) detecting second signals, or lack thereof, from the substrate during or subsequent to at least a subset of the second plurality of extension steps to determine second sequencing reads corresponding to the plurality of concatemers.

Clause 44. The method of clause 43, further comprising processing the sequencing reads and the second sequencing reads as paired end reads.

Clause 45. The method of any one of clauses 43-44, wherein the plurality of concatemers are amplified via amplification primers covalently bound to the substrate.

Clause 46. The method of any one of clauses 43-44, wherein the plurality of concatemers are amplified via amplification primers that are not covalently bound to the substrate.

Clause 47. The method of any one of clauses 1-46, wherein the plurality of concatemers are covalently bound to the substrate.

Clause 48. The method of any one of clauses 1-46, wherein the plurality of concatemers are not covalently bound to the substrate.

Clause 49. The method of clause 48, wherein the plurality of concatemers is bound to the substrate via electrostatic attraction.

Clause 50. A method for paired end sequencing, comprising: hybridizing a first primer to a first primer binding site on a template molecule; extending the first primer through a first region of the template molecule, wherein the extending comprises alternatively adding nucleotides and detecting incorporation of nucleotides; extending the first primer through a second region of the template molecule, thereby producing a copied template molecule, wherein the extending comprises adding nucleotides of at least one base type and, at one or more time points, not detecting incorporation of nucleotides; denaturing the copied template molecule from the template molecule; hybridizing a second primer to a second primer binding site on the copied template molecule; and extending the second primer through a first region of the copied template molecule, wherein the extending comprises alternatively adding nucleotides and detecting incorporation of nucleotides.

Clause 51. The method of clause 50, further comprising (g) extending the second primer through a second region of the copied template molecule, wherein the extending comprises adding nucleotides of at least one base type and, at one or more time points, not detecting incorporation of nucleotides.

Clause 52. The method of any one of clauses 50-51, wherein the extending (c) comprises, in one or more extension steps, adding nucleotides of two base types.

Clause 53. The method of any one of clauses 50-51, wherein the extending (c) comprises, in one or more extension steps, adding nucleotides of three base types.

Clause 54. The method of any one of clauses 50-51, wherein the extending (c) comprises, in one or more extension steps, adding nucleotides of four base types.

Clause 55. The method of any one of clauses 50-54 wherein a sequence of the first region of the template molecule is determined from detection of nucleotide incorporation in the extending of (b) and by at least one detection of nucleotide incorporation in the extending of (f).

Clause 56. The method of clause 55, wherein a sequence of the second region of the template molecule is determined from detection of nucleotide incorporation in the extending of (f) and by at least one detection of nucleotide incorporation in the extending of (b).

Clause 57. The method of any one of clauses 50-56, wherein each detection determines a base type of the respective incorporated nucleotide.

Clause 58. The method of clause 57, wherein each detection further comprises a confidence value of a respective nucleotide incorporation.

Clause 59. The method of any one of clauses 50-58, wherein the first primer binding site is at the 3′ end of the template molecule.

Clause 60. The method of any one of clauses 50-59, wherein the second primer binding site is at the 3′ end of the copied template molecule.

Clause 61. The method of any one of clauses 50-60, wherein the template molecule and the copied template molecule are each single-stranded.

Clause 62. The method of any one of clauses 50-61, wherein the nucleotides added during the extending (b) and (f) comprise reversibly terminated, labeled nucleotides.

Clause 63. The method of any one of clauses 51-62, wherein the nucleotides added during the extending (c) and (g) comprise a first subset of unlabeled nucleotides and a second subset of labeled nucleotides.

Clause 64. The method of any one of clauses 51-62, wherein the nucleotides added during the extending (c) and (g) comprise labeled nucleotides.

Clause 65. The method of any one of clauses 51-62, wherein the nucleotides added during the extending (c) and (g) comprise unterminated nucleotides.

Clause 66. The method of any one of clauses 50-65, wherein at least a subset of the nucleotides added during the extending (b) and (f) comprise unlabeled and/or unterminated nucleotides.

Clause 67. The method of any one of clauses 62-66, wherein the extending of (b) and (f) further comprise, after detecting incorporation of nucleotides, cleaving reversible terminators from incorporated nucleotides.

Clause 68. A method for loading concatemers on a substrate for sequencing, comprising: depositing a plurality of bead assemblies onto a substrate comprising a plurality of individually addressable locations, wherein a bead assembly of the plurality of bead assemblies comprises (i) a bead comprising surface primers and (ii) a circular template, wherein the circular template is bound to the bead via one of the surface primers, wherein the plurality of bead assemblies are immobilized on the plurality of individually addressable locations on the substrate; using the surface primers to amplify the circular template to generate a plurality of first stage concatemers and second stage concatemers immobilized to the substrate via the bead; and sequencing the first stage concatemers or the second stage concatemers immobilized to the substrate.

1-68. (canceled)

69. A method for sequencing on a functionalized surface, comprising:
(a) generating a layer comprising hydrophilic material on a surface of a substrate, wherein the layer comprises a plurality of attachment sites comprising a first reactive moiety;
(b) depositing a plurality of oligonucleotide molecules on the layer to immobilize the plurality of oligonucleotide molecules on the substrate at the plurality of attachment sites, wherein the plurality of oligonucleotide molecules comprises a second reactive moiety that reacts with the first reactive moiety; and
(c) depositing a plurality of nucleic acid molecules to the substrate to bind the plurality of nucleic acid molecules to oligonucleotide molecules of the plurality of oligonucleotide molecules immobilized on the substrate, thereby immobilizing the plurality of nucleic acid molecules on the substrate.

70. The method of claim 69, further comprising sequencing the plurality of nucleic acid molecules, or derivatives thereof, while immobilized on the substrate.

71. The method of claim 70, wherein the sequencing comprises rotating the substrate during dispensing of a sequencing reagent, during imaging, or both.

72. The method of claim 69, wherein (i) the oligonucleotide molecules are single-stranded primer molecules, (ii) nucleic acid molecules of the plurality of nucleic acid molecules are hybridized to the oligonucleotide molecules, and (iii) the method further comprises extending the single-stranded primer molecules.

73. The method of claim 72, further comprising amplifying the plurality of nucleic acid molecules while immobilized on the substrate to generate amplified molecules immobilized on the substrate.

74. The method of claim 69, further comprising, prior to (c), amplifying a plurality of library molecules to generate the plurality of nucleic acid molecules for the depositing in (c).

75. The method of claim 69, wherein the plurality of nucleic acid molecules is a plurality of DNA concatemers.

76. The method of claim 69, wherein the hydrophilic material comprises one or more members selected from the group consisting of: acrylic polymers, acrylonitrile polymers, acrylamide polymers, maleic anhydride polymers, ether polymers, polyesters, polyurethanes, polypropylene glycol, fluorocarbon polymers, polystyrene polymers, poly(vinyl chloride) (PVC) polymers, poly(n-vinylpyrrolidone) (PVP) polymers, polyethylene glycol (PEG), polyethylenimine, poly(acrylic acid), poly(sodium 4-styrenesulfonate), poly(allylamine hydrochloride), sodium polyacrylate, poly(vinyl sulfonic acid), poly(vinyl sulfate), poly(2-ethyl-2-oxazoline), polyacrylamide, poly(allylamine), poly(vinyl alcohol-co-ethylene), poly(4-styrenesulfonic acid-co-maleic acid), poly(acrylamide-co-acrylic acid), poly(2-(dimethylamino)ethyl methacrylate), poly(acrylamide-co-diallyldimethylammonium chloride), poly(styrene-alt-maleic acid), poly(2-dimethylamino)ethyl methacrylate) methyl chloride, poly(methyl vinyl ether), poly(methyl vinyl ether-alt-maleic acid), poly(N-isopropylacrylamide), poly(methacrylic acid), poly(N-isopropylacrylamide-co-methacrylic acid), poly(2-(diisopropylamino)ethyl methacrylate), polyvinyl alcohol (PVA), poly(N-isopropylacrylamide-co-butyl acrylate), poly(2-propyl-2-oxazoline), poly(2-methyl-2-oxazoline), poly(N-(2-hydroxypropyl) methacrylamide), poly(N,N-dimethylacrylamide), poly(N-isopropylacrylamide-co-acrylamide), poly(N-isopropylacrylamide), N-hydroxysuccinimide (NHS) ester, poly(2-propylacrylic acid), cucurbit[5]uril hydrate, cucurbit[7]uril hydrate, cucurbit[8]uril hydrate, poly(methyl vinyl ether-alt-maleic anhydride), poly(methyl vinyl ether-alt-maleic acid monoethyl ester), polyanetholesulfonic acid, poly[bis(2-chloroethyl) ether-alt-1,3-bis[3-(dimethylamino)propyl]urea] quaternized, and poly(4-styrenesulfonic acid.

77. The method of claim 76, wherein the hydrophilic material comprises PEG.

78. The method of claim 69, wherein the first reactive moiety and the second reactive moiety are azide and DBCO moieties, respectively, or DBCO and azide moieties, respectively.

79. The method of claim 69, wherein the first reactive moiety and the second reactive moiety react via a click chemistry reaction.

80. The method of claim 69, wherein the plurality of oligonucleotide molecules immobilized on the layer are not patterned.

81. The method of claim 69, wherein the surface of the substrate comprises silicon oxide.

82. A method for sequencing on a functionalized, comprising:
(a) providing a layered substrate, wherein the layered substrate comprises a first surface, a hydrophilic material layer deposited above the first surface, and a plurality of oligonucleotide molecules covalently bound to the hydrophilic material layer; and
(b) depositing a plurality of nucleic acid molecules to the layered substrate to bind the plurality of nucleic acid molecules to oligonucleotide molecules of the plurality of oligonucleotide molecules, thereby immobilizing the plurality of nucleic acid molecules on the layered substrate.

83. The method of claim 82, wherein the hydrophilic material comprises one or more members selected from the group consisting of: acrylic polymers, acrylonitrile polymers, acrylamide polymers, maleic anhydride polymers, ether polymers, polyesters, polyurethanes, polypropylene glycol, fluorocarbon polymers, polystyrene polymers, poly(vinyl chloride) (PVC) polymers, poly(n-vinylpyrrolidone) (PVP) polymers, polyethylene glycol (PEG), polyethylenimine, poly(acrylic acid), poly(sodium 4-styrenesulfonate), poly(allylamine hydrochloride), sodium polyacrylate, poly(vinyl sulfonic acid), poly(vinyl sulfate), poly(2-ethyl-2-oxazoline), polyacrylamide, poly(allylamine), poly(vinyl alcohol-co-ethylene), poly(4-styrenesulfonic acid-co-maleic acid), poly(acrylamide-co-acrylic acid), poly(2-(dimethylamino)ethyl methacrylate), poly(acrylamide-co-diallyldimethylammonium chloride), poly(styrene-alt-maleic acid), poly(2-dimethylamino)ethyl methacrylate) methyl chloride, poly(methyl vinyl ether), poly(methyl vinyl ether-alt-maleic acid), poly(N-isopropylacrylamide), poly(methacrylic acid), poly(N-isopropylacrylamide-co-methacrylic acid), poly(2-(diisopropylamino)ethyl methacrylate), polyvinyl alcohol (PVA), poly(N-isopropylacrylamide-co-butyl acrylate), poly(2-propyl-2-oxazoline), poly(2-methyl-2-oxazoline), poly(N-(2-hydroxypropyl) methacrylamide), poly(N,N-dimethylacrylamide), poly(N-isopropylacrylamide-co-acrylamide), poly(N-isopropylacrylamide), N-hydroxysuccinimide (NHS) ester, poly(2-propylacrylic acid), cucurbit[5]uril hydrate, cucurbit[7]uril hydrate, cucurbit[8]uril hydrate, poly(methyl vinyl ether-alt-maleic anhydride), poly(methyl vinyl ether-alt-maleic acid monoethyl ester), polyanetholesulfonic acid, poly[bis(2-chloroethyl) ether-alt-1,3-bis[3-(dimethylamino)propyl]urea] quaternized, and poly(4-styrenesulfonic acid.

84. The method of claim 83, wherein the hydrophilic material comprises PEG.

85. The method of claim 82, further comprising sequencing the plurality of nucleic acid molecules, or derivatives thereof, while immobilized on the layered substrate.

86. The method of claim 85, wherein the sequencing comprises rotating the layered substrate during dispensing of a sequencing reagent, during imaging, or both.

87. The method of claim 82, wherein (i) the oligonucleotide molecules are single-stranded primer molecules, (ii) nucleic acid molecules of the plurality of nucleic acid molecules are hybridized to the oligonucleotide molecules, and (iii) the method further comprises extending the single-stranded primer molecules.

88. The method of claim 82, further comprising amplifying the plurality of nucleic acid molecules while immobilized on the layered substrate to generate amplified molecules immobilized on the layered substrate.
